(19) United States
(12) Patent Application Publication
(10) Pub. No.: US 2016/0160252 A1
(43) Pub. Date: Jun. 9, 2016

(54) POLYMERS IN BIOMASS SACCHARIFICATION BIOPROCESS

(71) Applicant: EDENIQ, INC., Visalia, CA (US)

(72) Inventors: John Zhang, Camarillo, CA (US); Sandra Jacobson, El Cajon, CA (US); Kristoffer Ramos, Sanger, CA (US); Mrugesh Patel, Norton, VA (US); James Kacmar, Visalia, CA (US); Daniel Michalopoulos, Exeter, CA (US)

(21) Appl. No.: 14/907,212

(22) PCT Filed: Jul. 24, 2014
(86) PCT No.: PCT/US14/48003

§ 371 Date: Jan. 22, 2016

Related U.S. Application Data
(60) Provisional application No. 61/857,889, filed on Jul. 24, 2013.

(51) Int. Cl. C12P 19/14; C12P 19/02; C08F 126/10; C08F 116/06; C08G 65/08; C08G 65/34

(52) U.S. Cl. CPC C12P 19/14; C08G 65/08; C08G 65/34; C08F 126/10; C08F 116/06; C12P 19/02

(57) ABSTRACT

Methods and systems for increasing the yield of sugars from a biomass, such as a lignocellulosic biomass, are described. A non-ionic organic polymer is contacted with the biomass during the saccharification reaction, and the hydrolyzed mixture is separated using a filter into a permeate and a retentate, where the non-ionic organic polymer is present in the retentate. The retentate with the polymer is recycled to the hydrolysis mixture, which increased the yield of sugars using less saccharification enzymes. The methods thus allow for increased cost savings by reducing the amount of enzymes required to convert the biomass to sugars.

### CROSS-REFERENCE TO RELATED PATENT APPLICATIONS

The present application claims benefit of priority to U.S. Provisional Patent Application No. 61/857,889, filed Jul. 24, 2013, which is incorporated by reference herein in its entirety.

### BACKGROUND OF THE INVENTION

Biofuels such as ethanol can be produced from cellulosic biomass. While cellulosic ethanol production is currently possible, better efficiency in converting cellulosic biomass to biofuels will make the production of cellulosic biofuels more economically viable.

### BRIEF SUMMARY OF THE INVENTION

The present disclosure provides methods and systems for treating biomass, including a lignocellulosic biomass and/or a biomass comprising starch, to produce useful products such as carbohydrates and fermentable sugars. The biomass is treated with a non-ionic organic polymer that can be recovered and recycled to increase the yield of sugars from the biomass while reducing the amount of saccharification enzymes required. Thus, in one aspect, the disclosure provides methods for generating sugars from biomass, the method comprising:
(a) providing a mixture comprising the biomass, a non-ionic organic polymer of sufficient size to be captured by a filter; and one or more enzymes to hydrolyze components of the biomass to sugars;
(b) incubating the mixture under conditions such that the one or more enzymes hydrolyze components of the biomass to sugars, thereby producing a mixture of solids and a liquid comprising the polymer and sugars;
(c) separating the mixture into a liquid stream comprising the polymer and sugars, and a solids stream comprising solids;
(d) separating the liquid stream with the filter into a permeate comprising sugars and a retentate comprising the polymer; and
(e) returning at least a portion of the retentate to said mixture or a new mixture comprising biomass, thereby generating sugars and re-using the polymer.

In some embodiments, the polymer has the formula (I):

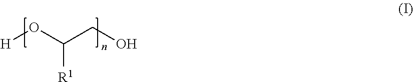

wherein R¹ is H, or a C₁₋₆ alkyl, and n is an integer greater than 1.

In some embodiments, the polymer has the formula (II):

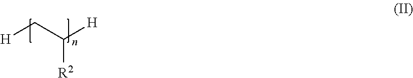

wherein R² is a hydroxyl, alkoxy, substituted or unsubstituted carboxylate, or substituted or unsubstituted heterocyclyl, and n is an integer greater than 1. In some embodiments, the alkoxy is a C₁₋₁₂alkoxy (e.g., methoxy). In some embodiments, the substituted or unsubstituted carboxylate is a C₁₋₆ carboxylate (e.g., —OC(O)CH₃). In some embodiments, the substituted or unsubstituted heterocyclyl is a pyrrolidone.

The mixture can comprise two or more different non-ionic organic polymers. In some embodiments, the two or more different non-ionic organic polymers comprise a polymer of formula (I) and a polymer of formula (II), wherein R¹ is H, or a C₁₋₆ alkyl, and n is an integer greater than 1, and R² is a hydroxyl, alkoxy, substituted or unsubstituted carboxylate, or substituted or unsubstituted heterocyclyl, and n is an integer greater than 1.

In some embodiments, the method further comprises returning at least a portion of the solids stream to the mixture, wherein the solids stream comprises at least a portion of the one or more enzymes. In some embodiments, the concentration of the polymer in the mixture is from about 0.1% to about 10.0% by weight of solids in the biomass.

The biomass can be a lignocellulosic biomass that is pretreated to make the biomass more accessible to hydrolytic enzymes. In some embodiments, the biomass comprises at least about 10% solids w/w added to the hydrolysis mixture.

The hydrolysis mixture can be separated into a liquid stream and a solids stream using a mechanical device, a filter, a membrane, or a tangential flow filtration device. In some embodiments, the mechanical device is a centrifuge, a press, or a screen.

The liquid stream can be passed through a filter to separate the liquid stream into a permeate comprising sugars, such as glucose and xylose, and a retentate comprising the polymer. In some embodiments, the filter comprises a membrane or a tangential flow filtration device.

In some embodiments, the biomass is treated with the polymer during the pretreatment step. In some embodiments, the biomass is treated with the polymer during the saccharification step. The methods of this aspect increase the yield of glucose and/or xylose when compared to methods that do not treat the biomass with a polymer during the pretreatment or saccharification steps.

The sugars produced by the method can be processed into ethanol, biofuels, biochemicals, or other chemical products. In some embodiments, the one or more enzymes comprises a cellulase, a hemicellulase, a β-glucosidase, and/or a xylanase.

In another aspect, a method for generating sugars from biomass is provided, the method comprising: contacting the biomass with a non-ionic organic polymer of sufficient size to be captured by a filter and one or more enzymes under conditions such that the one of more enzymes hydrolyze components of the biomass to sugars, thereby producing a mixture of solids and a liquid comprising the polymer and sugars. In some embodiments, the polymer has the formula (I):

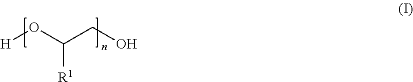

wherein R¹ is H, or a C₁₋₆ alkyl, and n is an integer greater than 1. In one embodiment, the polymer has the formula (II):

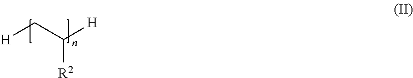

wherein R² is a hydroxyl, alkoxy, substituted or unsubstituted carboxylate, or substituted or unsubstituted heterocyclyl, and n is an integer greater than 1. In some embodiments, the alkoxy is a C₁₋₁₂alkoxy (e.g., methoxy). In some embodiments, the substituted or unsubstituted carboxylate is a C₁₋₆ carboxylate (e.g., —OC(O)CH₃). In some embodiments, the substituted or unsubstituted heterocyclyl is a pyrrolidone.

In some embodiments, n is greater than 25. In some embodiments, n is between 25 and 250,000. In some embodiments, the polymer of formula (I) has an average molecular weight or a viscosity average molecular weight (Mv) of from about 1,000 to about 10,000,000.

In the above aspects and embodiments, the temperature and pH range of the saccharification enzyme activity is expanded when compared to saccharification in the absence of a polymer described herein. For example, the activity of the enzyme(s) can be increased at temperatures that are higher than the optimal temperature for the enzyme activity. Thus, in some embodiments, the activity of the enzyme(s) is increased at temperatures higher than 55° C. compared to the activity of the enzyme(s) in the absence of the polymer of formula (I). In some embodiments, the activity of the enzyme(s) is increased at a pH of 6.0 compared to the activity of the enzyme(s) in the absence of the polymer of formula (I).

### DEFINITIONS

Unless defined otherwise, all technical and scientific terms used herein have the same meaning as commonly understood by one of ordinary skill in the art to which this invention pertains. Although essentially any methods and materials similar to those described herein can be used in the practice or testing of the present invention, only exemplary methods and materials are described. For purposes of the present invention, the following terms are defined below.

The terms “a,” “an,” and “the” include plural referents, unless the context clearly indicates otherwise.

The term “about,” when modifying any amount, refers to the variation in that amount typically encountered by one of skill in the art, i.e., in an ethanol production facility or testing lab. For example, the term “about” refers to the normal variation encountered in measurements for a given analytical technique, both within and between batches or samples. Thus, the term about can include variation of 1-10% of the measured value, such as 5% or 10% variation. The amounts disclosed herein include equivalents to those amounts, including amounts modified or not modified by the term “about.”

The term “catalyst” refers to a compound or substance that increases the rate of a chemical reaction, such as the hydrolysis of cellulose, or allows the reaction to proceed at substantially the same rate at a lower temperature. The term includes hydrolytic and saccharification enzymes that convert lignocellulosic biomass to polysaccharides, oligosaccharides, and/or simple fermentable sugars. The term also includes saccharification enzymes that are produced by genetically engineered or transgenic plants, for example, as described in U.S. Patent Publication 2012/0258503 to Rabb et al., which is incorporated by reference herein in its entirety. The term also includes polymeric acid catalysts, for example, as described in U.S. Patent Publications 2012/0220740, 2012/0252957, and 2013/0042859, which are each incorporated by reference herein in their entirety.

The term “biomass” refers to any material comprising lignocellulosic material. Lignocellulosic materials are composed of three main components: cellulose, hemicellulose, and lignin. Cellulose and hemicellulose contain carbohydrates including polysaccharides and oligosaccharides, and can be combined with additional components, such as protein and/or lipid. Examples of biomass include agricultural products such as grains, e.g., corn, wheat and barley; sugarcane; corn stover, corn cobs, bagasse, sorghum and other inedible waste parts of food plants; food waste; grasses such as switchgrass; and forestry biomass, such as wood, paper, board and waste wood products.

The term “lignocellulosic” refers to material comprising both lignin and cellulose, and may also contain hemicellulose.

The term “cellulosic,” in reference to a material or composition, refers to a material comprising cellulose.

The term “glucan” refers to all alpha and beta-linked 1,4, homopolymers of glucose subunits

The term “conditions suitable to hydrolyze components of the biomass to sugars” refers to contacting the solids phase biomass with one or more catalysts including, but not limited to, cellulase, hemicellulase and auxiliary enzymes or proteins in order to produce fermentable sugars from polysaccharides in the biomass. The conditions can further include a pH that is optimal for the activity of saccharification enzymes, for example, a pH range of about 4.0 to about 7.0. The conditions can further include a temperature that is optimal for the activity of catalysts, including saccharification enzymes, for example, a temperature range of about 35° C. to 75° C.

The term “hydrolysis” refers to breaking the glycosidic bonds in polysaccharides to yield simple monomeric and/or oligomeric sugars. For example, hydrolysis of cellulose produces the six carbon (C6) sugar glucose, whereas hydrolysis of hemicellulose produces the five carbon (C5) sugars including xylose and arabinose. Generating short chain cellulosic sugars from polymer cellulosic fibers and biomass can be achieved by a variety of techniques, processes, and or methods. For example, cellulose can be hydrolyzed with water to generate cellulosic sugars. Hydrolysis can be assisted and or accelerated with the use of hydrolytic enzymes, chemicals, mechanical shear, thermal and pressure environments, and or any combination of these techniques. Examples of hydrolytic enzymes include cellulases and hemicellulases and amylases. Cellulase is a generic term for a multi-enzyme mixture including exo-cellobiohydrolases, endoglucanases and β-glucosidases which work in combination to hydrolyze cellulose to cellobiose and glucose. Hydrolytic enzymes are also referred to as “saccharification enzymes.” Examples of non-hydrolytic enzymes include oxidoreductases such as manganese peroxidase and laccase, and lyases that assist in production of fermentable sugars. Examples of chemicals include strong acids, weak acids, weak bases, strong bases, ammonia, or other chemicals. Mechanical shear includes high shear orifice, cavitation, colloidal milling, and auger milling. Examples of high shear devices include an ICS-type orifice reactor (Buchen-Industrial Catalyst Service), a rotating colloidal-type mill, a Silverson mixer, cavitation milling device, or steam assisted hydro jet type mill.

The terms “high-shear agitation,” “high-shear mixing,” and “high-shear milling” refer to subjecting the biomass to conditions of high shear in order to reduce the biomass particle size. In some embodiments, the conditions produce a biomass particle size distribution from about 1 to about 800 microns. In some embodiments, the biomass particle size distribution is such that at least about 70%, 75%, 80%, 85%, 90%, or 95% of the particles have a size of from about 1 to about 800 microns, from about 2 to about 600 microns, from about 2 to about 400 microns, or from about 2 to about 200 microns. High-shear conditions can be provided by devices well known in the art, for example, by an ICS-type orifice reactor (Buchen-Industrial Catalyst Service), a rotating colloidal-type mill, a Silverson mixer, cavitation milling device, or steam assisted hydro jet type mill.

The term “saccharification” refers to production of fermentable sugars from biomass or biomass feedstock. Saccharification can be accomplished by catalysts including hydrolytic enzymes described herein and/or auxiliary proteins, including, but not limited to, peroxidases, laccases, expansins and swollenins.

The term “fermentable sugar” refers to a sugar that can be converted to ethanol or other products such as butanols, propanols, succinic acid, and isoprene, during fermentation, for example during fermentation by yeast. For example, glucose is a fermentable sugar derived from hydrolysis of cellulose, whereas xylose, arabinose, mannose and galactose are fermentable sugars derived from hydrolysis of hemicellulose.

The term “simultaneous saccharification and fermentation” (SSF) refers to providing saccharification enzymes during the fermentation process. This is in contrast to the term “separate hydrolysis and fermentation” (SHF) steps.

The term “pretreatment” refers to treating the biomass with physical, chemical or biological means, or any combination thereof, to render the biomass more susceptible to hydrolysis, for example, by saccharification enzymes. Pretreatment can comprise treating the biomass at elevated pressures and/or elevated temperatures. Pretreatment can further comprise physically mixing and/or milling the biomass in order to reduce the size of the biomass particles. Devices that are useful for physical pretreatment of biomass include, e.g., a hammermill, shear mill, cavitation mill or colloid or other high-shear mill. An exemplary colloid mill is the Cellunator™ (Edeniq, Visalia, Calif.). Reduction of particle size is described in, for example, WO2010/025171, which is incorporated by reference herein in its entirety.

The term “elevated pressure,” in the context of a pretreatment step, refers to a pressure above atmospheric pressure (e.g., 1 atm at sea level) based on the elevation, for example at least 20, 30, 40, 50, 60, 70, 80, 90, 100, 110, 120, 130, 140, or 150 psi or greater at sea level.

The term “elevated temperature,” in the context of a pretreatment step, refers to a temperature above ambient temperature, for example at least 100, 110, 120, 130, 140, 150, 160, 170, 180, 190, or 200 degrees C. or greater. When used in hydrothermal pretreatment, the term includes temperatures sufficient to substantially increase the pressure in a closed system. For example, the temperature in a closed system can be increased such that the pressure is at least 100 psi or greater, such as 110, 120, 130, 140, 150 psi or greater.

The term “pretreated biomass” refers to biomass that has been subjected to pretreatment to render the biomass more susceptible to hydrolysis.

The term “non-ionic organic polymer” refers to any neutrally charged synthetic or naturally occurring long chain molecule consisting of repeating units of one or more carbon-containing monomers or building units.

The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, undecyl, dodecyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol.

The term “alkoxy” refers to an alkyl group bound through a single, terminal ether linkage; that is, an “alkoxy” group can be defined as —OZ¹ where Z¹ is alkyl as defined above.

The term “cycloalkyl” refers to a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocyclyl” is a cycloalkyl group as defined above where at least one of the carbon atoms of the ring is substituted with a heteroatom such as, but not limited to, nitrogen, oxygen, sulfur, or phosphorus. The cycloalkyl group and heterocyclyl group can be substituted or unsubstituted. The cycloalkyl group and heterocyclyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol.

The term “carboxylate” or “carboxyl” group refers to a group represented by the formula —C(O)O⁻.

The term “hydroxyl” refers to a group represented by the formula —OH.

The term “sulfonate” refers to the sulfo-oxo group represented by the formula —S(O)₃⁻.

The term “solid/liquid separation” refers to methods by which a solids fraction is separated from a liquids stream using mechanical devices such as but not limited to centrifuges, presses, screens; settling tanks, flotation cells, cyclone cleaners, sieves, and the like.

The term “membrane type separation” refers to methods by which a liquid stream is partitioned into separate streams using mechanical devices such as but not limited to ultrafiltration (UF) membranes, microfiltration (MF) membranes, and Tangential Flow Filtration (TFF) systems.

The term “recycle” refers to the return of material such as liquids, solids, polymers or enzymes to a previous stage in a cyclic or continuous process.

The term “PEG” refers to polyethylene glycol, which is an oligomer or polymer of ethylene oxide. The term PEG is chemically synonymous with polyethylene oxide (PEO) and polyoxyethylene (POE). Thus, as used herein, the term PEG is sometimes used interchangeably with PEO and POE.

### BRIEF DESCRIPTION OF THE DRAWINGS

FIG. 1 shows a representative embodiment of the methods described herein.

FIG. 2 shows the glucose yield (%, w/v) from pretreated bagasse that was treated with different molecular weights of PEO during the saccharification reaction. The glucose yield was determined after 48 hours of saccharification (3% by weight of dry mass).

FIG. 3 shows the percentage increase in glucose yields based on the data in FIG. 1, where the control yield without PEO treatment represents 100%.

FIG. 4 shows the xylose yield (%, w/v) from bagasse treated as in FIG. 1.

FIG. 5 shows the glucose yield (%, w/v) and percent increase from pretreated bagasse hydrolyzed in a mixture comprising 3% PEO at different pH (pH 4.0, 5.0 and 6.0). The glucose yield was determined after 24 (T24) and 48 (T48) hours of saccharification. The percent increase was calculated using the glucose concentration in w/v % from the pH5.0/0 PEO experiment as a baseline.

FIG. 6 shows the xylose yield (%, w/v) and percent increase from bagasse treated as in FIG. 5.

FIG. 7 shows the glucose yield (%, w/v) from pretreated bagasse treated with 3% PEO at different temperatures (50° C., 55° C., and 60° C.) at different time points (4, 8, 24 and 48 hours) during the saccharification reaction.

FIG. 8 shows the xylose yield (%, w/v) from bagasse treated as in FIG. 7.

FIG. 9 shows the glucose yield (%, w/v) from pretreated bagasse that was treated with 2% of recycled retentate during the saccharification reaction, where the saccharification reactions comprised different amounts of enzyme loading (20%, 15%, and 10% Accellerase® Trio™ (Trio) based on glucan content). The glucose yield was determined after 24 (T24) and 48 (T48) hours of saccharification, and compared to saccharification reactions that were not treated with retentate (negative controls), or were treated with 3% PEG (positive control).

FIG. 10 shows the glucose yield (%) from the data in FIG. 9, where 20% enzyme loading and no retentate was set at 100%.

FIG. 11 shows the glucose yield (%, w/v) from corn stover pretreated with 3.0% PEG at different time points of saccharification.

FIG. 12 shows the xylose yield (%, w/v) from corn stover pretreated with 3.0% PEG at different time points of saccharification.

FIG. 13 shows the glucose yield (%, w/v) from bagasse treated with different concentrations of PVP during saccharification.

FIG. 14 shows the xylose yield (%, w/v) from bagasse treated with different concentrations of PVP during saccharification.

FIG. 15 shows the glucose yield (%, w/v) from bagasse treated with different molecular weights of PVP during saccharification.

FIG. 16 shows the xylose yield (%, w/v) from bagasse treated with different molecular weights of PVP during saccharification.

FIG. 17 shows the percentage increase in glucose and xylose yields from bagasse treated with different molecular weights of PVP during saccharification (no PVP control=100%).

FIG. 18 shows the glucose yield (%, w/v) from bagasse treated with PVP and PEG. HPHT stands for High Pressure High Temperature pretreatment. HPHT+PVP=PVP added during saccharification. HPHT/PVP=PVP added during pretreatment. HPHT/PVP+PEG=PVP added during pretreatment and PEG added during saccharification.

FIG. 19 shows the xylose yield (%, w/v) from bagasse treated with PVP and PEG. Abbreviations as in FIG. 18.

FIG. 20 shows the percentage increase in glucose and xylose conversion rate from bagasse treated with PVP and PEG after 24 (left two columns) and 48 (right two columns) hours of saccharification. Abbreviations as in FIG. 18.

FIG. 21 shows the percentage increase in glucose and xylose yields from bagasse treated with PVP and PEG after 24 (left two columns) and 48 (right two columns) hours of saccharification. Abbreviations as in FIG. 18.

FIG. 22 shows the glucose and xylose yields (%, w/v) from bagasse treated with PVP and different amounts of saccharification enzymes.

FIG. 23 shows polymers having a polyvinyl structure that were tested for improved saccharification efficiency, as described in the Examples.

FIG. 24 shows the glucose yield (%, w/v) from bagasse treated with different polymers during saccharification.

FIG. 25 shows the xylose yield (%, w/v) from bagasse treated with different polymers during saccharification.

FIG. 26 shows the glucose yield (%, w/v) from acid-pretreated corn stover that was treated with PVP during saccharification.

FIG. 27 shows the glucose and xylose yields (%, w/v) from pretreated swithgrass that was treated with PVP during the pretreatment step (HPHT/2% PVP) or during the saccharification step (HPHT+2% PVP).

FIG. 28 shows the glucose and xylose yields (%, w/v) from pretreated almond shell biomass that was treated with PVP during saccharification.

FIG. 29 shows a representative embodiment of a system as described herein.

### DETAILED DESCRIPTION OF THE INVENTION

### Introduction

The present disclosure provides methods and systems for treating biomass, including a lignocellulosic biomass and/or a biomass comprising starch, to produce useful products such as carbohydrates and fermentable sugars. The methods described herein unexpectedly increase the conversion of cellulosic biomass to sugars by treating the biomass with a non-ionic organic polymer before or during the hydrolysis step. In particular, the methods increase the yield of sugars produced from the biomass while at the same time reducing the amount of saccharification enzymes required for hydrolyzing cellulose to sugars, when compared to methods known in the art. The methods can also increase the conversion rate of biomass to sugars, when compared to methods known in the art. The methods are also useful for increasing the amount of non-ionic organic polymer that is recovered and available for recycling. The recovered non-ionic organic polymer can be used to increase the conversion rate of biomass to sugars and/or reduce the amount of saccharification enzymes required for hydrolyzing cellulose to sugars. The methods of the disclosure will now be described.

### I. Methods

The methods described herein are useful for increasing the yield of sugars from biomass, such as a lignocellulosic biomass or a biomass comprising starch. The methods typically comprise treating the biomass in a mixture comprising a non-ionic organic polymer and one or more hydrolytic enzymes in order to hydrolyze components of the biomass to sugars. In certain embodiments, the non-ionic organic polymer is of sufficient size to be captured by a filter. The hydrolysis mixture is incubated under conditions suitable for the enzymes to hydrolyze components of the biomass to sugars, the hydrolysis producing a mixture comprising solids and a liquid comprising the polymer and sugars. The mixture can then be separated into a liquid stream comprising the polymer and sugars, and a solids stream comprising solids. In some embodiments, the liquid stream is then separated into a permeate comprising sugars and a retentate comprising the polymer. In one embodiment, the liquid stream is separated into a permeate and a retentate using a filter, such as a membrane or Tangential Flow Filtration (TFF) system. The filter can be selected such that the polymer is retained in the retentate, and the sugars (e.g., glucose and/or xylose) flow through with the permeate. The retentate or a portion thereof can be recycled and returned to the original hydrolysis mixture, or can be added to a new hydrolysis mixture. The new hydrolysis mixture can comprise fresh biomass and one or more enzymes, and optionally can comprise fresh non-ionic organic polymer. In some embodiments, the retentate with the recycled polymer is added to a new hydrolysis mixture without adding additional or fresh non-ionic organic polymer, thereby reducing the amount of polymer required.

In some embodiments, the non-ionic organic polymer is a polymer of ethylene oxide, such as polyethylene glycol (PEG). PEG is also referred to as polyethylene oxide (PEO) or polyoxyethylene (POE), depending on its molecular weight. Historically, PEG referred to oligomers and polymers with a molecular mass below 20,000 g/mol, PEO to polymers with a molecular mass above 20,000 g/mol, and POE to a polymer of any molecular mass. However, in the Examples and Figures described herein, the terms PEG, PEO, and POE are used interchangeably.

In some embodiments, the non-ionic organic polymer is polypropylene glycol. In some embodiments, the non-ionic organic polymer has the structure of formula (I):

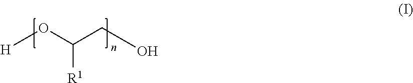

wherein R¹ is H, or a C₁₋₆ alkyl, and n is an integer greater than 1.

In some embodiments, the non-ionic organic polymer comprises a polyvinyl structure, such as polyvinylpyrrolidone (PVP), a PVP co-polymer (Poly (1-vinylpyrrolidone-co-vinyl acetate), PVE (Poly (methyl vinyl ether)), or PVA (Polyvinyl alcohol). In some embodiments, the non-ionic organic polymer has the structure of formula (II):

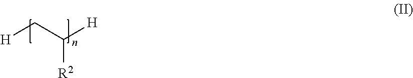

wherein R² is a hydroxyl, alkoxy, substituted or unsubstituted carboxylate, or substituted or unsubstituted heterocyclyl, and n is an integer greater than 1. In some embodiments, the non-ionic organic polymer is PVP, a PVP co-polymer, PVE, or PVA.

In certain embodiments, the mixture can comprise two or more different non-ionic organic polymers. For example, in one embodiment, the mixture comprises a polymer of formula (I), wherein R¹ is H, or a C₁₋₆ alkyl, and n is an integer greater than 1, and a polymer of formula (II), wherein R² is a hydroxyl, alkoxy, substituted or unsubstituted carboxylate, or substituted or unsubstituted heterocyclyl, and n is an integer greater than 1. In some embodiments, the alkoxy is a C₁₋₁₂alkoxy (e.g., methoxy). In some embodiments, the substituted or unsubstituted carboxylate is a C₁₋₆ carboxylate (e.g., —OC(O)CH₃). In some embodiments, the substituted or unsubstituted heterocyclyl is a pyrrolidone.

In some embodiments, Formula II can be represented by one or more of the following structures:

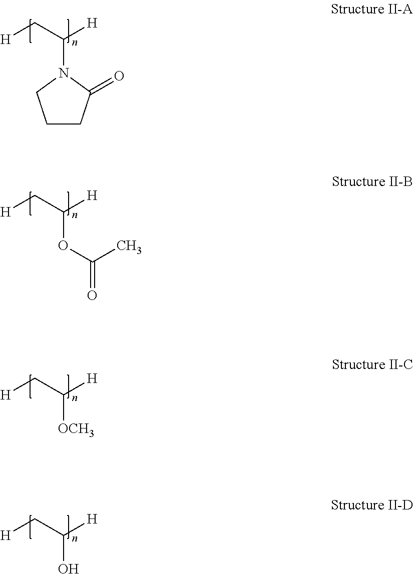

In some embodiments, n is greater than 25. In some embodiments, n is between about 25 and 250,000. In some embodiments of the method, the polymer has an average molecular weight or a viscosity average molecular weight (Mv) of from about 1,000 to about 10,000,000. For example, the average molecular weight or the Mv of the polymer can be at least about 1K, 2K, 5K, 10K, 20K, 30K, 40K, 50K, 100K, 200K, 300K, 400K, 500K, 1,000,000 (1M), 2M, 3M, 4M, 5M, 6M, 7M, 8M, 9M, or 10M.

In some embodiments, the size of the non-ionic organic polymer is sufficient to be retained by a filter. Thus, the size of non-ionic organic polymer can be selected to be large enough to be retained in the retentate, and still have the desired properties of increasing the yield of fermentable sugars during a saccharification reaction.

In some embodiments, the concentration of the polymer in the mixture is from about 0.1% to about 10.0% by weight of solids in the biomass. For example, the concentration of the polymer in the mixture can be about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0% by weight of solids in the biomass. In some embodiments, the concentration of the polymer in the mixture is greater than about 10.0% by weight of solids in the biomass.

The one or more enzymes used in the methods can include cellulases, hemicellulases, β-glucosidase, and xylanase.

The method can further comprise returning or recycling the solids stream, or a portion thereof, to the hydrolysis mixture. The solids stream can comprise the saccharification enzymes that were added to the original mixture. While not being limited by theory, it is believed that the enzymes adsorb to the surface of the dissolved solids. Thus, in some embodiments, the solids stream comprises at least a portion of the one or more saccharification enzymes added to the hydrolysis mixture.

In some embodiments, the biomass is a lignocellulosic biomass. In some embodiments, the biomass comprises at least about 5%, at least about 7%, at least about 10%, at least about 15%, at least about 20%, at least about 25%, or at least about 30% solids w/w when added to the hydrolysis mixture. In some embodiments, the biomass comprises from about 5% to about 30% or more solids w/w when added to the hydrolysis mixture. It will be understood that any range described herein includes the end points of the range and any point in between.

In some embodiments, the biomass is a pretreated biomass. Methods for pretreating biomass are described in more detail herein. In some embodiments, the non-ionic organic polymer can be added to the biomass during the pretreatment step.

In some embodiments, the mixture is separated into a liquid stream and solids stream using any suitable separation method or device known in the art. For example, the mixture can be separated into a liquid stream and solids stream using a mechanical device, a filter, a membrane, or a tangential flow filtration (TFF) device. Non-limiting examples of mechanical devices include centrifuges, presses, or screens.

The methods described herein increase the yield of glucose and/or xylose when compared to a hydrolysis mixture that does not comprise a non-ionic organic polymer described herein. The sugars produced by the method can be processed into ethanol, biofuels, biochemicals, or other chemical products, as known in the art. Specific embodiments of the method for increasing the yield of glucose and/or xylose by using a non-ionic organic polymer described herein are described in the Examples.

In another aspect, the disclosure provides a method for generating sugars from biomass, where the method comprises contacting the biomass with a non-ionic organic polymer of sufficient size to be captured by a filter and one or more enzymes under conditions such that the one or more enzymes hydrolyze components of the biomass to sugars. The hydrolysis produces a mixture of solids and a liquid, the liquid comprising the polymer and sugars.

In some embodiments of this aspect of the disclosure, the non-ionic organic polymer has the structure of formula (I):

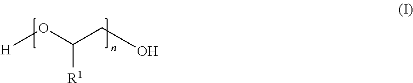

wherein R¹ is H, or a C₁₋₆ alkyl, and n is an integer greater than 25.

In some embodiments, n is between about 25 and 250,000. In some embodiments of the method, the polymer has the structure of formula (I), and has an average molecular weight or a viscosity average molecular weight (Mv) of from about 1,000 to about 10,000,000. For example, the average molecular weight or the Mv of the polymer can be at least about 1K, 2K, 5K, 10K, 20K, 30K, 40K, 50K, 100K, 200K, 300K, 400K, 500K, 1,000,000 (1M), 2M, 3M, 4M, 5M, 6M, 7M, 8M, 9M, or 10M.

Surprisingly, the methods described herein increased the conversion rate of biomass to glucose at temperatures above the optimum activity range for the hydrolytic enzymes. Thus, the addition of a non-ionic organic polymer described herein to the hydrolysis mixture can extend the temperature range of the saccharification enzymes. For example, in any of the above aspects and embodiments, the method increases the activity of the one or more saccharification enzymes at temperatures greater than 55° C. as compared to the activity of the one or more enzymes in the absence of a non-ionic organic polymer described herein. The increase in glucose yield also occurs at temperatures within the optimum range for the enzymes, as described in the Examples. In some embodiments, the increase in enzyme activity produces 10%, 15%, 20%, 25%, 30%, 35%, 40%, 45%, 50% or more glucose yield when compared to the amount of glucose produced in the absence of a non-ionic organic polymer under identical conditions using the same amount of enzyme activity (e.g., the same amount of the same enzyme(s) or an equivalent amount of different enzymes having the same enzymatic activity) at the same saccharification temperature.

In some embodiments, the methods increased the activity of the saccharification enzymes at a higher pH than is optimal for the enzymes. For example, in some embodiments, the activity of the one or more enzymes is increased at a pH of 6.0 compared to the activity of the one or more enzymes in the absence of the polymer.

The methods described herein provide the following unexpected advantages. First, adding a non-ionic organic polymer to the hydrolysis mixture increased the saccharification rate of the biomass by at least 20%. Second, the inventors found that the non-ionic organic polymer can be recovered using a filter system, and that the recovered polymer improved the saccharification efficiency similar to or better than fresh (unrecycled) polymer. Third, adding recycled polymer to the hydrolysis mixture can decrease the amount of fresh enzyme required by about 50%. Fourth, the cellulase enzyme beta-glucosidase can also be recovered by the filter system, and recycled with the polymer to increase saccharification efficiency and lower enzyme costs. Fifth, the addition of the polymer makes the saccharification conditions more flexible in that the temperature and pH ranges are broader than in the absence of the polymer. Sixth, the increased saccharification efficiencies provided by the methods are applicable to a broad range of biomass substrates, such as bagasse, pine wood chips and corn stover.

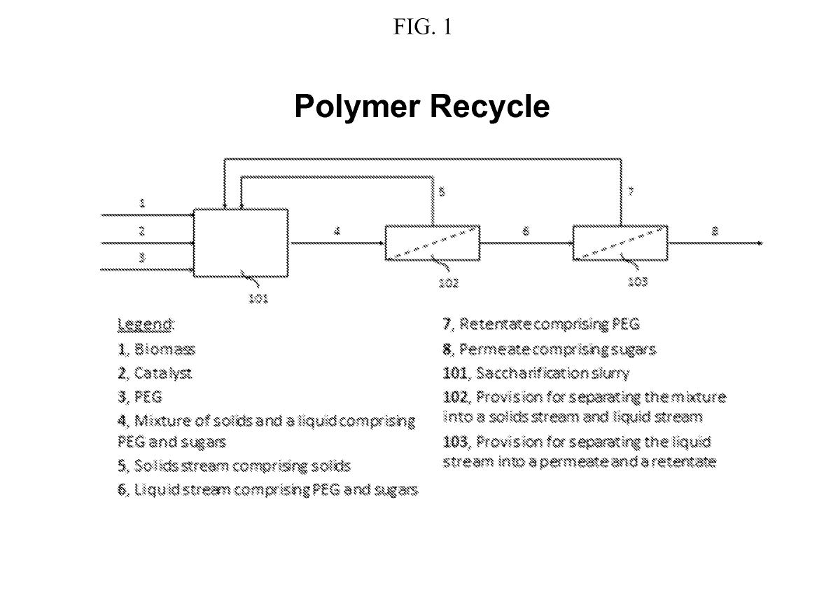

The methods described herein can be batch, semi-batch, or continuous. A flow chart illustrating one non-limiting representative embodiment is shown in FIG. 1. As shown in FIG. 1, Biomass (1), catalyst (2) and a polymer such as PEG (3) are added to a saccharification slurry (101). After hydrolysis of the biomass, the hydrolyzed mixture (4) is separated (102) into a solids stream (5) and a liquid stream comprising the PEG and sugars (6). The liquid stream (6) is separated (103) into a retentate comprising PEG (7) and a permeate comprising sugars (8). The solids stream (5) and retentate (7) can be recycled back to the saccharification slurry (101). The permeate (8) can be processed to produce a biofuel such as ethanol or other downstream products.

### A. Pretreatment

Prior to the hydrolysis steps described herein, the biomass can be pretreated to render the lignocellulose and cellulose more susceptible to hydrolysis. Pretreatment includes treating the biomass with physical, chemical or biological means, or any combination thereof, to render the biomass more susceptible to hydrolysis, for example, by saccharification enzymes. Examples of chemical pretreatment are known in the art, and include acid pretreatment and alkali pretreatment.

One example of physical pretreatment includes elevated temperature and elevated pressure. Thus, in some embodiments, pretreatment comprises subjecting the biomass to elevated temperatures and elevated pressure in order to render the lignocellulose and cellulose accessible to enzymatic hydrolysis. In some embodiments, the temperature and pressure are increased to amounts and for a time sufficient to render the cellulose susceptible to hydrolysis. In some embodiments, the pretreatment conditions can comprise a temperature in the range of about 150° C. to about 210° C. The pretreatment temperature can be varied based on the duration of the pretreatment step. For example, for a pretreatment duration of about 60 minutes, the temperature is about 160 degrees C.; for a duration of 30 minutes, the temperature is about 170 degrees C.; for a duration of 5 minutes, the temperature is about 210 degrees C.

The pretreatment conditions can also comprise increased pressure. For example, in some embodiments, the pressure can be at least 100 psi or greater, such as 110, 120, 130, 140, 150 psi or greater. In some embodiments, the biomass is pretreated in a closed system, and the temperature is increased in an amount sufficient to provide the desired pressure. In one embodiment, the temperature is increased in the closed system until the pressure is increased to about 125 to about 145 psi. Persons of skill in the art will understand that the temperature increase necessary to increase the pressure to the desired level will depend on various factors, such as the size of the closed system. In some embodiments, pretreatment comprises any other method known in the art that renders lignocellulose and cellulose more susceptible to hydrolysis, for example, acid treatment, alkali treatment, and steam treatment, or combinations thereof.

In some embodiments, the pretreatment step does not result in the production of a substantial amount of sugars. For example, in some embodiments, pretreatment results in the production of less than about 10%, 5%, 1%, 0.1%, 0.01%, or 0.001% by weight glucose, less than about 10%, 5%, 1%, 0.1%, 0.01%, or 0.001% by weight xylose, and/or less than about 10%, 5%, 1%, 0.1%, 0.01%, or 0.001% by weight sugars in general. In some embodiments, the amount of sugars in the process stream entering the pretreatment stage is substantially the same as the amount of sugars in the process stream exiting the pretreatment stage. For example, in some embodiments, the difference between the amount of sugars in the process stream entering the pretreatment stage and the amount of sugars exiting the pretreatment stage is less than about 10%, 5%, 1%, 0.1%, 0.01%, or 0.001% by weight.

In some embodiments, pretreatment can further comprise physically mixing and/or milling the biomass in order to reduce the size of the biomass particles. The yield of biofuel (e.g., ethanol) can be improved by using biomass particles having relatively small sizes. Devices that are useful for physical pretreatment of biomass include, e.g., a hammermill, shear mill, cavitation mill or colloid or other high shear mill. Thus, in some embodiments, the pretreatment step comprises physically treating biomass with a colloid mill. An exemplary colloid mill is the Cellunator™ (Edeniq, Visalia, Calif.). In some embodiments, the biomass is physically pretreated to produce particles having a relatively uniform particle size of less than about 1600 microns. For example, at least about 50%, 60%, 70%, 80%, 85%, 90%, or 95% of the pretreated biomass particles can have a particle size from about 100 microns to about 800 microns. In some embodiments, at least about 50%, 60%, 70%, 80%, 85%, 90%, or 95% of the pretreated biomass particles have a particle size from about 100 microns to about 500 microns. In some embodiments, the biomass is physically pretreated to produce particles having a relatively uniform particle size using a colloid mill. The use of a colloid mill to produce biomass particles having a relatively uniform particle size, e.g., from about 100 microns to about 800 microns, can result in increased yield of sugars, as described in U.S. Patent Application Publication 2010/0055741 (Galvez et al.), which is incorporated by reference herein in its entirety.

In some embodiments, the biomass or a mixture comprising biomass and an aqueous fluid such as water is pretreated with a high shear milling or mixing device comprising a rotor and a stator, wherein the high shear milling or mixing device has a gap setting between the rotor and stator of between about 0.1 and about 1.2 mm. For example, the gap setting can be about 0.1, 0.2, 0.3, 0.4, 0.5, 0.6, 0.7, 0.8, 0.9, 1.0, 1.1, or 1.2 mm, including values between each indicated value. Pretreatment using the indicated gap setting reduces the size of biomass particles, rendering a greater percentage of the biomass available for conversion to sugars, e.g., by enzymes, as compared to pretreatment of the biomass with a hammer mill alone. In some embodiments, the gap between the rotor and stator is adjustable.

In one embodiment, the high shear milling or mixing device is a colloid mill. Commercial colloid mills have a gap setting that can be dynamically adjusted to accommodate subtle differences in each biofuel plant including the percent backset, type of centrifuge or other particle separation process equipment, and other factors. The colloidal mill can be used to select the resulting particle size distribution through the use of gap rotational controls. A relatively precise particle size distribution can be obtained from much larger biomass material using a colloid mill in contrast to alternative pretreatment techniques such as comminution with a hammer mill. An appropriate gap size on the colloid mill can produce a highly uniform suspension of biomass, where the maximum particle size of the biomass is greatly reduced and significantly more uniform compared to using only the comminution device. The radial gap size for a colloidal mill used in a corn ethanol plant can range from about 0.104-0.728 millimeters, e.g., from about 0.104-0.520 millimeters, e.g., from about 0.208-0.520 millimeters, such that the resulting particle sizes are in the range of about 100-800 microns. For example, in some embodiments, a gap setting of about 0.1-0.15 is used for corn stover or other cellulosic biomass and a gap setting of about 0.2-0.3 mm is used for grains including but not limited to corn kernels. The use of a colloid mill to produce relatively precise, uniform particles sizes with high surface area results in a greater percent of starch, cellulose and sugar being available for enzymatic conversion than a hammer mill, leading to improved yield.

Typically, the finer the biomass the better the attained yield with respect to gallons of biofuel per ton of biomass. However, a serious overriding factor in the overall process is the recovery of residual solids after the biofuel has been removed. This factor results in an optimal biomass size of 100-500 microns for corn ethanol. For cellulosic processes that utilize rice straw, sugar cane, energy cane and other materials where state of the art filtration equipment can be installed, biomass particle size can be from about 50-350 microns, typically from about 75-150 microns. In some embodiments, the biomass is contacted with cellulosic enzymes before the biomass is pretreated with the high shear milling or mixing device. In some embodiments, the biomass is contacted with cellulosic enzymes after the biomass is pretreated with the high shear milling or mixing device.

In some embodiments, the pretreatment step does not involve the use of acids which can degrade sugars into inhibitors of fermentation.

In some embodiments, the pH of the pretreated biomass is adjusted to a pH of between about 3.0 and about 6.5. In some embodiments, the pH of the biomass is adjusted during or after the pretreatment step to be within the optimal range for activity of saccharification enzymes, e.g., within the range of about 4.0 to 6.0. In some embodiments, the pH of the biomass is adjusted using Mg(OH)₂, NH₄OH, NH₃, or a combination of Mg(OH)₂ and NH₄OH or NH₃.

After pretreatment, the pretreated biomass is hydrolyzed to produce sugars using the methods described herein.

### B. Separation Methods and Devices

The methods described herein make use of various types of separators and separation methods. In some embodiments, the separator is a screen type separator. Non-limiting examples of screen type separators include screens, vibrating screens, reciprocating screens (rake screens), gyratory screens/sifters, and pressure screens. In some embodiments, separator is capable of separating solids from liquids. Non-limiting examples of solid/liquid separators include mechanical devices such as but not limited to centrifuges, presses, screens; settling tanks, flotation cells, cyclone cleaners, sieves, and the like.

In some embodiments, the separator is a membrane type separator. Examples of membrane type separators include ultrafiltration (UF) membranes, microfiltration (MF) membranes, and Tangential Flow Filtration (TFF) systems.

MF membranes typically have a pore size of between 0.1 micron and 10 microns. Examples of microfiltration membranes include glass microfiber membranes such as Whatman GF/A membranes. UF membranes have smaller pore sizes than MF membranes, typically in the range of 0.001 to 0.1 micron. UF membranes are typically classified by molecular weight cutoff (MWCO). Examples of ultrafiltration membranes include polyethersulfone (PES) membranes having a low molecular weight cutoff, for example about 10 kDa. UF membranes are commercially available, for example from Synder Filtration (Vacaville, Calif.).

Filtration using either MF or UF membranes can be employed in direct flow filtration (DFF) or Tangential Flow Filtration (TFF). DFF, also known as dead end filtration, applies the feed stream perpendicular to the membrane face such that most or all of the fluid passes through the membrane. TFF, also referred to as cross-flow filtration, applies the feed stream parallel to the membrane face such that one portion passes through the membrane as a filtrate or permeate whereas the remaining portion (the retentate) is recirculated back across the membrane or diverted for other uses. TFF filters include microfiltration, ultrafiltration, nanofiltration and reverse osmosis filter systems. The cross-flow filter may comprise multiple filter sheets (filtration membranes) in a stacked arrangement, e.g., wherein filter sheets alternate with permeate and retentate sheets. The liquid to be filtered flows across the filter sheets, and solids or high-molecular-weight species of diameter larger than the filter sheet's pore size(s), are retained and enter the retentate flow, whereas the liquid along with any permeate species diffuse through the filter sheet and enter the permeate flow. The TFF filter sheets, including the retentate and permeate sheets, may be formed of any suitable materials of construction, including, for example, polymers, such as polypropylene, polyethylene, polysulfone, polyethersulfone, polyetherimide, polyimide, polyvinylchloride, polyester, etc.; nylon, silicone, urethane, regenerated cellulose, polycarbonate, cellulose acetate, cellulose triacetate, cellulose nitrate, mixed esters of cellulose, etc.; ceramics, e.g., oxides of silicon, zirconium, and/or aluminum; metals such as stainless steel; polymeric fluorocarbons such as polytetrafluoroethylene; and compatible alloys, mixtures and composites of such materials. Cross-flow filter modules and cross-flow filter cassettes useful for such filtration are commercially available from SmartFlow Technologies, Inc. (Apex, N.C.). Suitable cross-flow filter modules and cassettes of such types are variously described in the following United States patents: U.S. Pat. No. 4,867,876; U.S. Pat. No. 4,882,050; U.S. Pat. No. 5,034,124; U.S. Pat. No. 5,034,124; U.S. Pat. No. 5,049,268; U.S. Pat. No. 5,232,589; U.S. Pat. No. 5,342,517; U.S. Pat. No. 5,593,580; and U.S. Pat. No. 5,868,930; the disclosures of all of which are hereby incorporated herein by reference in their respective entireties.

In some embodiments, the filter is a TFF filter having a molecular weight size limit suitable to retain a non-ionic organic polymer described herein in the retentate. It will be understood that filters with lower molecular weight size limits should result in a higher recovery of the polymer. However, the lower molecular weight size limits are expected to result in slower filtration rates, such that the optimum molecular weight size limit will represent a trade-off between polymer recovery and flow-through rates. In some embodiments, the filter has a 150 kDa membrane.

In some embodiments, the separator is a reverse osmosis (RO) type separator. Examples of RO type separators include RO spiral membranes available from Koch Membrane Systems (Wilmington, Mass.) or Synder Filtration (Vacaville, Calif.).

### C. Saccharification and Fermentation Conditions

The saccharification reaction can be performed at or near the temperature and pH optimum for the saccharification enzymes used. In some embodiments of the present methods, the temperature optimum for saccharification ranges from about 15 to about 100° C. In other embodiments, the temperature range is about 20 to 80° C., about 35 to 65° C., about 40 to 60° C., about 45 to 55° C., or about 45 to 50° C. The pH optimum for the saccharification enzymes can range from about 2.0 to 11.0, about 4.0 to 6.0, about 4.0 to 5.5, about 4.5 to 5.5, or about 5.0 to 5.5, depending on the enzyme.

Examples of enzymes that are useful in saccharification of lignocellulosic biomass include glycosidases, cellulases, hemicellulases, starch-hydrolyzing glycosidases, xylanases, ligninases, and feruloyl esterases, and combinations thereof. Glycosidases hydrolyze the ether linkages of di-, oligo-, and polysaccharides. The term cellulase is a generic term for a group of glycosidase enzymes which hydrolyze cellulose to glucose, cellobiose, and other cello-oligosaccharides. Cellulase can include a mixture comprising exo-cellobiohydrolases (CBH), endoglucanases (EG) and β-glucosidases (BG). Specific examples of saccharification enzymes include carboxymethyl cellulase, xylanase, β-glucosidase, β-xylosidase, and α-L-arabinofuranosidase, and amylases. Saccharification enzymes are commercially available, for example, Pathway™ (Edeniq, Visalia, Calif.), Cellic® CTec2 and HTec2 (Novozymes, Denmark), Spezyme® CP cellulase, Multifect® xylanase, and Trio® (Genencor International, Rochester, N.Y. Saccharification enzymes can also be expressed by host organisms, including recombinant microorganisms.

The enzyme saccharification reaction can be performed for a period of time from about several minutes to about 250 hours, or any amount of time between. For example, the saccharification reaction time can be about 5 minutes, 10 minutes, 30 minutes, 60 minutes, or 2, 4, 6, 8, 12, 16, 18, 24, 36, 48, 60, 72, 84, 96, 108, 120, 130, 140, 150, 160, 170, 180, 190, 200, 210, 220, 230, 240 or 250 hours. In other embodiments, the saccharification reaction is performed with agitation to improve access of the enzymes to the cellulose.

The amount of saccharification enzymes added to the reaction can be adjusted based on the cellulose content of the biomass and/or the amount of solids present in a composition comprising the biomass, and also on the desired rate of cellulose conversion. For example, in some embodiments, the amount of enzymes added is based on percent by weight of cellulose present in the biomass, as specified by the enzyme provider(s). The percent of enzyme added by weight of cellulose in such embodiments can range, for example, from about 0.1% to about 10% on this basis.

After the biomass is pretreated and hydrolyzed as described herein, the sugars can be used for any desired downstream process or refined as a product. In one embodiment, the sugars are fermented to ethanol, as described below.

After the saccharification steps described above, the treated biomass and/or converted sugars can be subjected to fermentation under conditions sufficient to produce ethanol from the sugars. The fermentation conditions include contacting the biomass and/or sugars with yeast that are capable of producing ethanol from sugars. If desired, the biomass can be subjected to simultaneous saccharification and fermentation (SSF). The pH of the SSF reaction can be maintained at the optimal ranges for the activity of the cellulosic enzymes, for example between about 4.0 and 6.0, or between about 4.5 and 5.0.

In some embodiments, the fermentation process contains particles in a fluid mash, and the downstream process further comprises separating the particles from the residual fluid mash using separation equipment. In some embodiments, a high-shear mixing device is used to produce particles with a relatively uniform particle size as described herein consistent for use with the separation equipment. In one embodiment, a colloid mill having gap rotational controls for choosing a gap size is used to choose a gap size to produce particles with a relatively uniform particle size consistent for use with the separation equipment.

### II. Polymers

The methods described herein use a non-ionic organic polymer. In some embodiments, the non-ionic organic polymer is PEG or PEO. In some embodiments, the non-ionic organic polymer is a compound having the structure of formula (I):

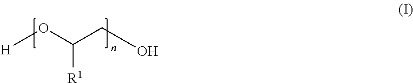

wherein R¹ is H, or a C₁₋₆ alkyl, and n is an integer greater than 1.

In some embodiments, the non-ionic organic polymer comprises a polyvinyl structure, such as polyvinylpyrrolidone (PVP), a PVP co-polymer (Poly (1-vinylpyrrolidone-co-vinyl acetate), PVE (Poly (methyl vinyl ether)), or PVA (Polyvinyl alcohol). In some embodiments, the non-ionic organic polymer is a compound having the structure of formula (II):

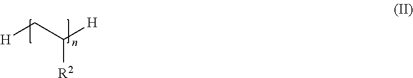

wherein R² is a hydroxyl, alkoxy, substituted or unsubstituted carboxylate, or substituted or unsubstituted heterocyclyl, and n is an integer greater than 1.

In some embodiments, the polymer comprises monomers selected from the following group:

### Vinyl-pyrrolidone

### N-Vinyl-caprolactam

### N-Vinyl-imidazole

Methyl vinyl ether
Ethyl vinyl ether
n-Butyl vinyl ether
iso-Butyl vinyl ether
Cyclohexyl vinyl ether
2-Ethylhexyl vinyl ether
1,4-Butanediol divinyl ether
Diethyleneglycol divinyl ether
Hydroxybutyl vinyl ether
Vinyl acetate

### Acrylamide

In some embodiments, the polymer comprises polymers selected from the following group:

Poly (vinyl acetate)
Poly (vinyl alcohol)
Poly (vinyl alcohol-co-ethylene)
Poly (vinyl alcohol-co-vinyl acetate)
Poly (vinyl pyrrolidone)
Poly (vinyl pyrrolidone-co-vinyl acetate)
Poly (vinyl pyrrolidone-co-vinyl alcohol)
Poly (vinyl pyrrolidone-co-styrene)
Poly (methyl vinyl ether)
Poly (acrylamide)

### Poly (N-isopropylacrylamide)

Poly (N-isopropylacrylamide-co-acrylamide)
Poly (2-hydroxyethyl methacrylate)
Polyethylene glycol
Polyethylene oxide
Poly (ethylene glycol) diacrylamide
Poly (ethylene glycol) methyl ether-block-poly (D,L) lactide
Poly (styrene)-block-poly (ethylene glycol)
Poly (ethylene glycol-ran-propylene glycol)
Polyethylene oxide dendrimers

### III. Systems

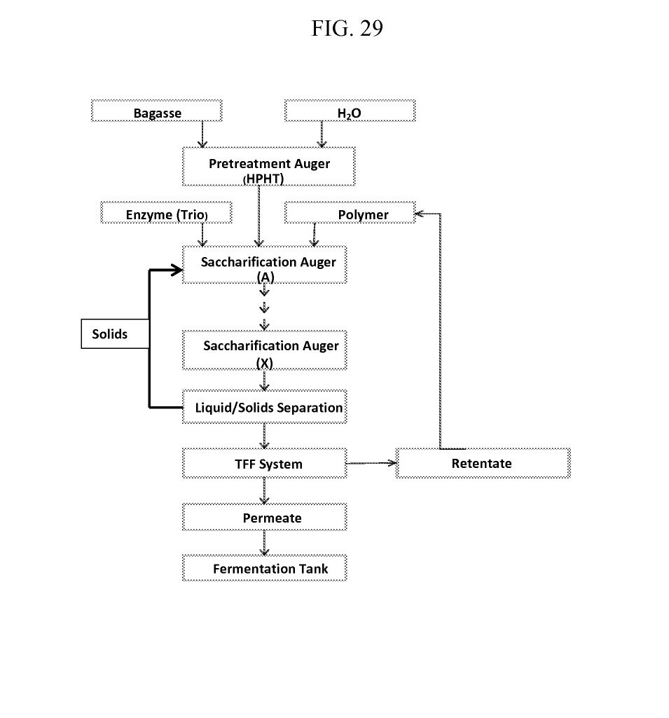

In another aspect, a system is described that uses the methods described herein. As shown in the representative embodiment illustrated in FIG. 29, the system comprises a continuous saccharification system in fluid connection with a TFF membrane system. In operation of the system, cellulosic biomass, such as bagasse, is mixed with an aqueous fluid (such as H₂O) and subjected to HPHT pretreatment. The HPHT pretreatment can occur in a high shear mixing device such as an auger. After pretreatment, the pretreated biomass is hydrolyzed in a saccharification reactor. The saccharification reactor can be a high shear mixing device, such as an auger. Saccharification enzymes and a non-ionic organic polymer, such as PEO or PVP, is added to the saccharification mixture. If desired, a series of saccharification reactors in fluid connection can be used (labeled (A) to (X), where X is an integer). In some embodiments, the system further comprises a solid/liquid separation system or device in fluid connection with a saccharification reactor. Suitable solid/liquid separation systems or devices are described herein, and include, without limitation, centrifuges, presses, screens, and settling tanks. However, any suitable solid/liquid separation system or device known in the art can be used. Following saccharification, the liquefied biomass is separated into a liquid stream and a solids stream using the solid/liquid separation system or device. The solids stream (“solids”) can be recycled back to a saccharification reactor (A)-(X) that is in fluid connection with the solid/liquid separation system or device The solids can further comprise enzymes that are recycled back to a saccharification reactor, where the recycled enzymes increase the efficiency of saccharification and reduce the amount of fresh enzymes that are required for saccharification, thereby reducing the cost of fresh enzymes. In some embodiments, the system comprises a TFF system in fluid connection with the solid/liquid separation system or device. The liquid stream from the solid/liquid separation system or device is contacted with the TFF system, which separates the liquid stream into a retentate and permeate. The permeate comprising sugars can be sent to a fermentation tank in fluid communication with the TFF system for the production of ethanol. Alternatively, the permeate and sugars can be used for any desired downstream purpose. In some embodiments, the TFF system is in fluid communication with a saccharification reactor for recycling the retentate back to a saccharification reactor. The recycled retentate comprises the non-ionic organic polymer and enzymes, which further improves the saccharification efficiency and reduces the amount of fresh enzymes required, providing a cost savings to the ethanol plant operator.

As will be understood by one of skill in the art, the system described herein can be operated in a batch, a fed batch, or a continuous manner.

### EXAMPLES

The following examples are offered to illustrate, but not to limit the claimed invention.

### Example 1

This example demonstrates that treatment of bagasse with PEO during the saccharification step increased the amount of glucose produced.

Methods:

Bagasse containing 27% glucan and 15% xylan was pretreated at 175° C. for 30 minutes and 10% solids loading. Following pretreatment, the bagasse biomass slurry was contacted with enzymes (20% by weight Accellerase® Trio™ based on glucan content of the biomass) and 3% PEO (by weight based on dry solids loaded). The resulting mixture was subjected to saccharification for 48 hours at 50° C. Samples were removed at times T0 and T24 for HPLC analysis to measure sugar and inhibitor concentrations.

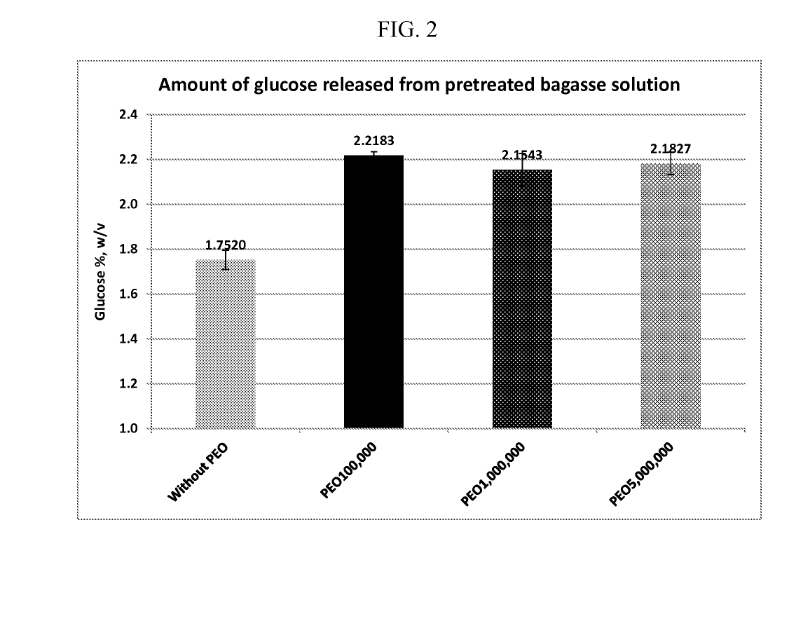

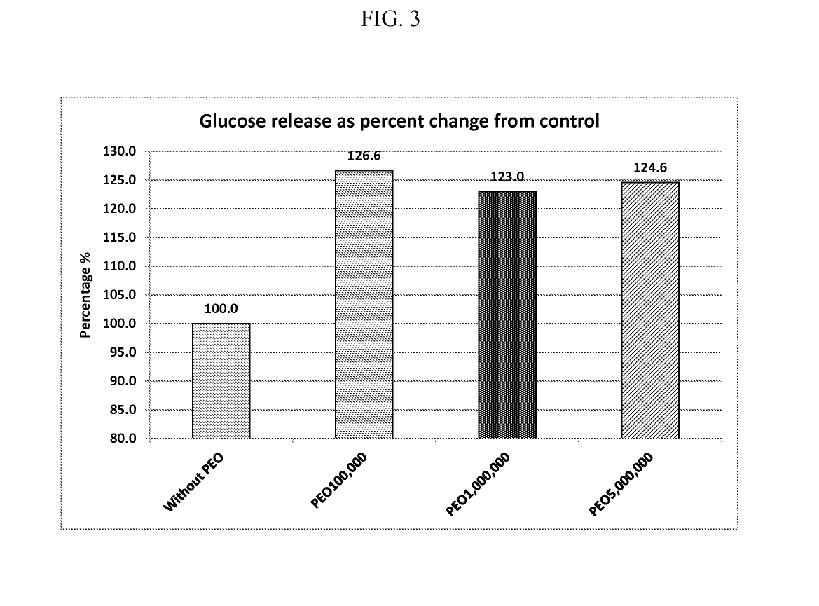

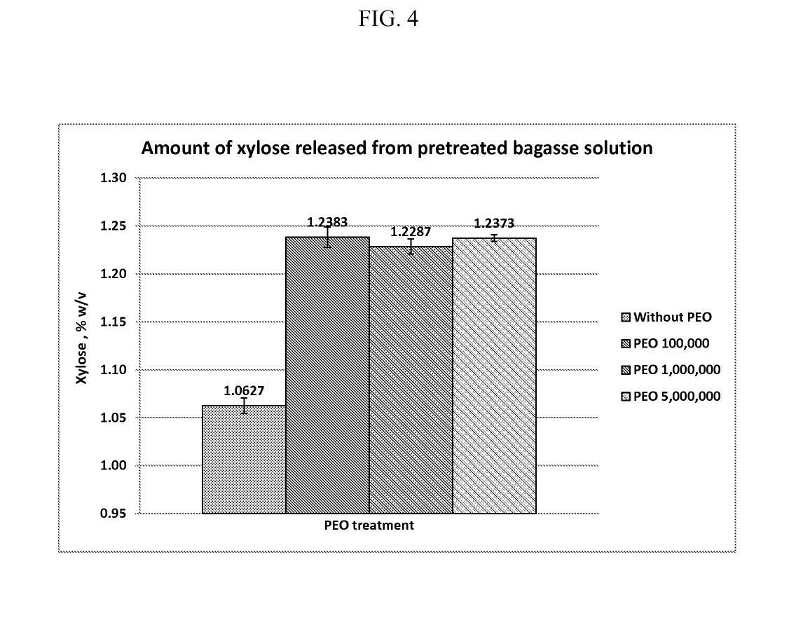

As shown in FIG. 2, treatment with PEO polymers of different molecular weights (100,000; 1,000,000; 5,000,000) increased the amount of glucose (%, w/v) produced compared to bagasse hydrolyzed in the absence of PEO. FIG. 3 shows that all three PEO polymers tested increased the percentage of glucose released from the bagasse by about 23 to 26%. FIG. 4 shows that treatment with all three PEO polymers tested increased the amount of xylose (%, w/v) released compared to bagasse hydrolyzed in the absence of PEO.

This example shows that pretreated bagasse contacted with PEO during the hydrolysis step resulted in greater than a 23% increase in glucose produced, and an increase in the amount of xylose produced. This example also shows that PEO polymers having different molecular weights resulted in a similar increase in the amount of sugars released during the hydrolysis step.

### Example 2

This example demonstrates that PEO can increase the conversion rate of glucan to glucose at different pH.

Methods:

Bagasse comprising 40.7% glucan and 22.7% xylan in a 15% solids slurry was pretreated in a one liter bomb reactor at 175° C. for 30 minutes. The pH of the pretreated material was adjusted to pH 4.0, 5.0 and 6.0 in different flasks. The pretreated bagasse was contacted with enzymes (20% by weight Accellerase® Trio™ based on glucan content of the biomass) and 3% PEO 1,000,000 (by weight based on dry solids loaded). The resulting mixture was subjected to saccharification for 48 hours at 50° C. Samples were removed at times T24 and T48 for HPLC analysis to measure sugar and inhibitor concentrations.

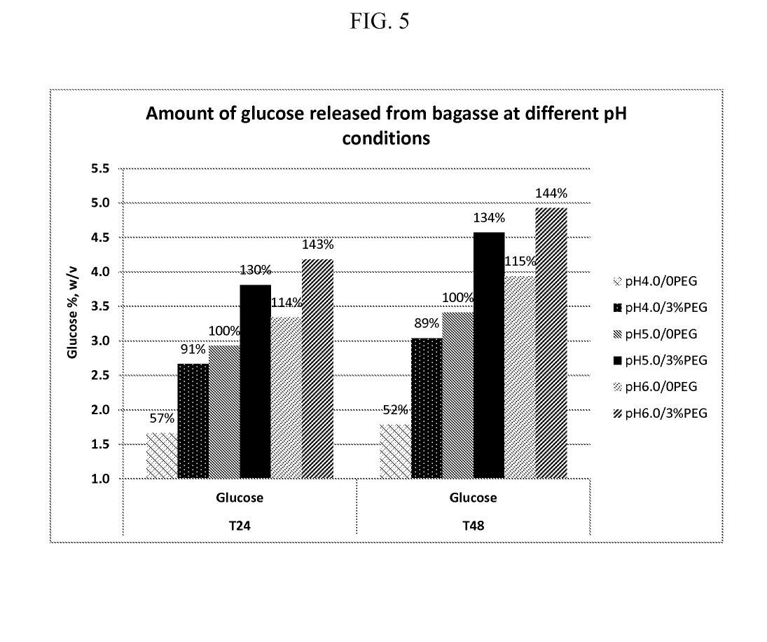

As shown in FIG. 5, PEO treatment resulted in an increase in the amount of glucose released (%, w/v) from the pretreated bagasse at each pH tested, compared to a control. The increase in the amount of glucose released was observed at both 24 and 48 hour time points. The percent increase was calculated using the glucose concentration in % w/v from the pH5.0/0 PEO experiment as a baseline (see Table 1 below).

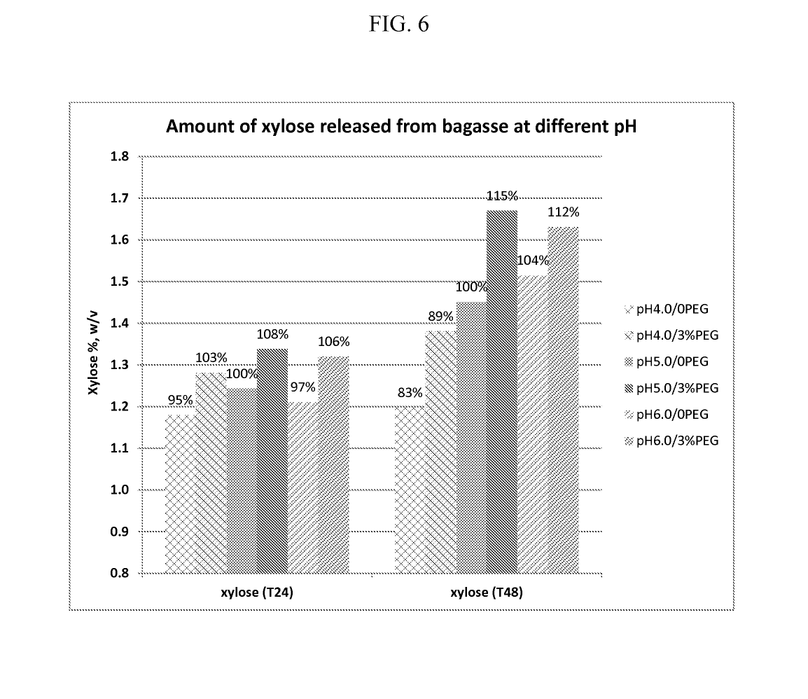

FIG. 6 shows that PEO treatment also resulted in an increase in the amount of xylose released (%, w/v) from the pretreated bagasse at each pH tested, compared to a control. The increase in the amount of xylose released was more pronounced at the 48 hour time point.

As shown in Table 1, PEO treatment also increased the glucose conversion rate at each pH tested.

TABLE 1
Glucose conversion rate of glucan in pretreated bagasse
with and without PEO treatment at different pH.

| | T24 | | T48 | |
|---|---|---|---|---|
| | Glucose | Conv. % | Glucose | Conv. % |
| pH 4.0/0PEO | 1.6649 | 25.2 | 1.7861 | 27.1 |
| pH 4.0/3% PEO | 2.6667 | 40.4 | 3.0393 | 46.1 |
| PH 5.0/0PEO | 2.9284 | 44.4 | 3.4120 | 51.7 |
| pH 5.0/3% PEO | 3.8108 | 57.7 | 4.5684 | 69.2 |
| pH 6.0/0PEO | 3.3414 | 50.6 | 3.9371 | 59.7 |
| pH 6.0/3% PEO | 4.1826 | 63.4 | 4.9273 | 74.7 |

Table 2 shows that treatment with PEO did not significantly affect inhibitor levels.

TABLE 2
Inhibitors levels from pretreated bagasse with and without PEO
treatment at different pH. Values shown are mg/liter or ppm.

| | T24 (Sacc) | | | | | | | | |
|---|---|---|---|---|---|---|---|---|---|
| | Furolic | | | | Syringic | | | Coumaric | Ferulic |
| | Acid | 5-HMF | Furfural | 4HBA | Acid | Vanillin | Syringaldehyde | Acid | Acid |
| pH 4/0% PEO | 76.2 | 314.6 | 2345.4 | 58.7 | 29.2 | 54.1 | 24.8 | 20.7 | 5.9 |
| pH 4/3% PEO | 77.0 | 323.8 | 2410.4 | 64.0 | 31.2 | 57.9 | 62.6 | 22.7 | 10.0 |
| pH 5/0% PEO | 88.3 | 348.1 | 2552.9 | 62.0 | 39.2 | 58.8 | 159.1 | 22.3 | 17.2 |
| pH 5/3% PEO | 88.0 | 348.8 | 2584.4 | 61.7 | 40.6 | 60.2 | 146.2 | 24.1 | 16.9 |
| pH 6/0% PEO | 78.1 | 337.7 | 2558.5 | 60.0 | 42.5 | 55.8 | 183.3 | 21.9 | 19.9 |
| pH 6/3% PEO | 78.0 | 339.6 | 2612.6 | 60.0 | 42.3 | 57.2 | 154.6 | 23.8 | 18.8 |

This example demonstrates that PEO treatment resulted in a 30-40% increase in glucose released from pretreated bagasse, and that the increase occurred at three different pH levels. Treatment with PEO at pH 6.0 resulted in the greatest increase in glucose. The glucose conversion rate was also increased, with 74.7% of the glucan converted to glucose at pH 6.0 and 3% PEO treatment after 48 hours. Importantly, the increase in sugars produced by PEO treatment did not result in an increase in inhibitor levels.

### Example 3

This example demonstrates that PEO can increase the conversion rate of glucan to glucose at different temperatures.

Methods:

Dried bagasse was adjusted to 15% solids (w/w) and pretreated at 175° C. for 30 minutes. The pH was adjusted to pH 5.0, and the solids was re-adjusted to 15%. The material was treated with enzymes and PEO as described in Example 2. Saccharification was performed in 100 gram samples using 500 mL Erlenmeyer flasks and incubated at the following temperatures: 50°, 55°, and 60° C. for 48 hours. Samples were measured via HPLC at T=4, T=8, T=24, and T=48 using the C5 Sugars method.

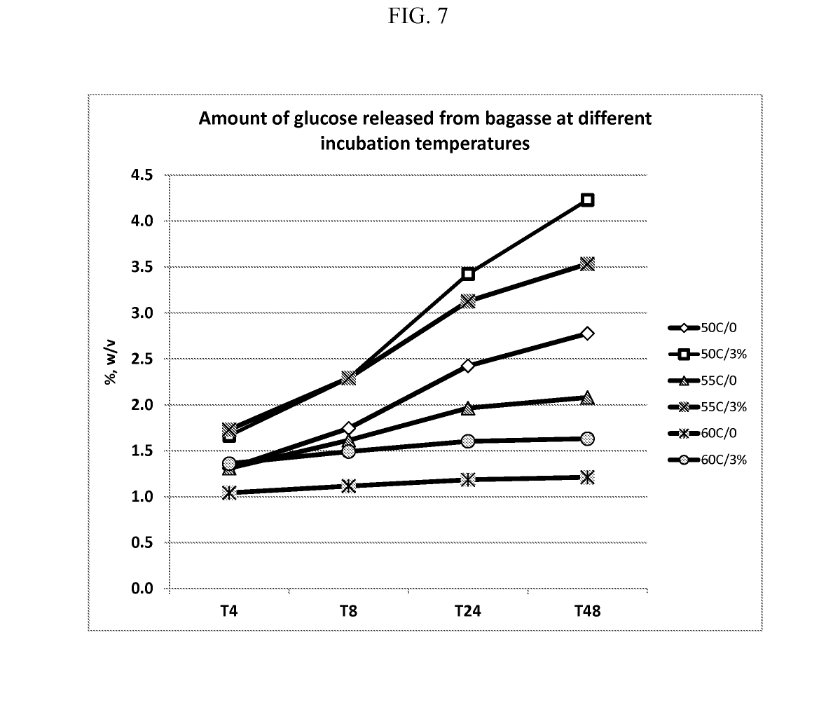

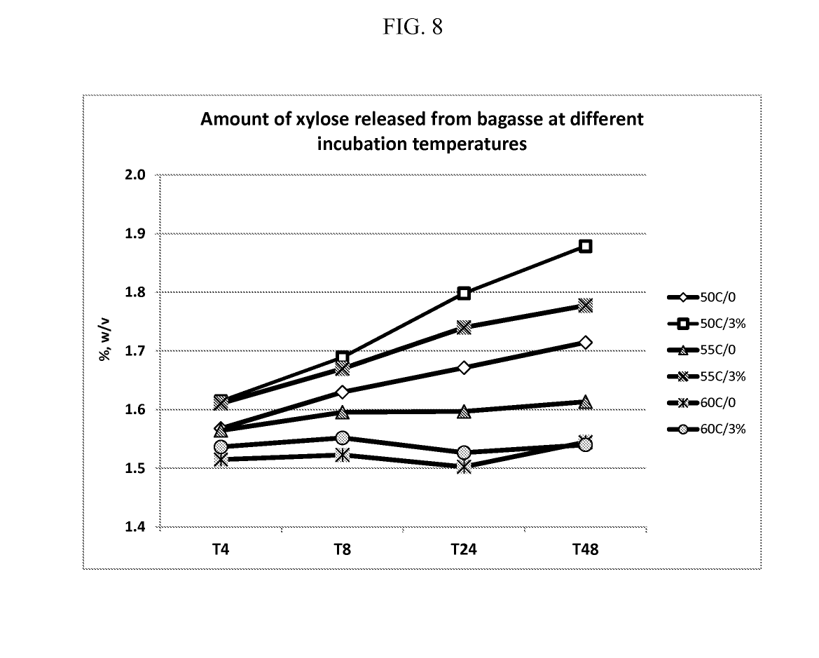

As shown in FIG. 7, treatment with PEO increased the amount of glucose obtained at all three temperatures tested. Similarly, treatment with PEO increased the amount of xylose obtained, though the effect was less pronounced at 60° C. (FIG. 8). Table 3 shows the conversion rate to glucose and xylose at 50°, 55°, and 60° C., with and without PEO, at four different time points.

TABLE 3
Glucose and xylose conversion rates at different temperatures with and without PEO treatment*.

| | T4 | | T8 | | T24 | | T48 | |
|---|---|---|---|---|---|---|---|---|
| | Glucose % | Xylose % | Glucose % | Xylose % | Glucose % | Xylose % | Glucose % | Xylose % |
| 50° C./0 PEO | 100.0 | 100.0 | 100.0 | 100.0 | 100.0 | 100.0 | 100.0 | 100.0 |
| 50° C./3% PEO | 127.1 | 103.0 | 131.3 | 103.6 | 141.3 | 107.6 | 152.4 | 109.6 |
| 55° C./0 PEO | 100.1 | 99.8 | 92.6 | 97.9 | 81.1 | 95.5 | 74.9 | 94.1 |
| 55° C./3% PEO | 132.1 | 102.7 | 131.4 | 102.5 | 129.0 | 104.1 | 127.3 | 103.7 |
| 60° C./0 PEO | 79.6 | 96.6 | 64.0 | 93.4 | 48.9 | 89.9 | 43.6 | 90.1 |
| 60° C./3% PEO | 103.9 | 98.0 | 85.6 | 95.2 | 66.1 | 91.3 | 58.7 | 89.8 |

*50° C. and no PEO treatment was set at 100% for each time point.

The data shows that the addition of PEO increased the conversion rate of glucan to glucose at 50° C. by about 27% after 4 hours of saccharification, and that the conversion rate increased to about 52% after 48 hours of saccharification. The data also shows that, at 55° C., the conversion rate of glucan to glucose was increased by about 32% at 4 hours of saccharification, and that the conversion rate increased to about 52% after 48 hours of saccharification.

Importantly, this example also demonstrates that PEO increased the glucose conversion rate at temperatures above the optimum range for the saccharification enzymes used (see, e.g., Product Information from Genencor®, which shows a rapid decline in activity above about 50° C.). For example, as shown in Table 3, above 55° C. the activity of the Accellerase® Trio™ enzymes declines (compare 60° C., no PEO, to 55° C., no PEO, and note that the relative conversion rate decreases at 60° C. over time, from 79.6% to 43.6% relative to 50° C., indicating that the enzymes are losing activity at the higher temperature). However, in the presence of PEO, the conversion rate at 60° C. increased by about 24% at T4, and by about 15% at T48.

In summary, this example shows that PEO increased the conversion rate of biomass to glucose at all temperatures tested, and suggests that PEO extended the temperature range of the saccharification enzymes used.

### Example 4

This example shows that PEO can be recycled to increase the saccharification efficiency of pretreated bagasse.

Methods:

Bagasse (11% solids) was pretreated at 175° C. for 30 minutes. Saccharification was performed as described in Example 2, with Accellerase® Trio™ at 20% loading based on 33% assumed glucan content. The saccharification reaction included 3% PEG3500. Following saccharification at 50° C. for 48 hours, a sample was removed and centrifuged at 9000 rpm for 30 minutes. The supernatant was passed through a TFF system with a 150 kDa membrane. All the fraction samples were analyzed by HPLC to measure sugar and inhibitor levels.

As shown in Table 4, the retentate contained only about 0.1% of the amount of glucose and xylose present in the feed material, whereas the permeate contained the majority of the sugars.

TABLE 4
Amounts of sugar and acetic acid recovered in the
different TFF fractions. Values shown are w/v %.

| | | T0 | | | |
|---|---|---|---|---|---|
| Sample# | Treatment | Glucose | Xylose | Arabinose | Acetic acid |
| 1 | Feed | 2.2880 | 1.6677 | 0.0902 | 0.3643 |
| 2 | Retentate | 0.1046 | 0.0717 | 0.0019 | 0.0143 |
| 3 | Permeate | 2.0700 | 1.5090 | 0.0832 | 0.3282 |
| 4 | wash solution | 0.3424 | 0.2466 | 0.0153 | 0.0501 |

As shown in Table 5, the retentate also contained substantially less inhibitors than the permeate.

TABLE 5
Inhibitor amounts recovered in the different TFF fractions. Values shown are mg/liter or ppm.

| | | T0 | | | | | | | | |
|---|---|---|---|---|---|---|---|---|---|---|
| | | Furolic | | | | Syringic | | | Coumaric | Ferulic |
| Sample# | Treatment | acid | 5-HMF | Furfural | 4HBA | acid | Vanillin | Syringaldehyde | acid | acid |
| 1 | Feed | 21.9 | 28.5 | 350.5 | 55.6 | 20.7 | 57.3 | 102.3 | 27.9 | 82.1 |
| 2 | Retentate | 1.0 | 1.3 | 16.3 | 4.2 | 3.4 | 5.7 | 7.9 | 4.4 | 2.0 |
| 3 | Permeate | 19.8 | 25.3 | 284.1 | 46.8 | 17.0 | 47.9 | 91.4 | 25.0 | 74.7 |
| 4 | wash | 3.2 | 4.2 | 56.1 | 9.5 | 5.5 | 11.6 | 17.1 | 6.3 | 11.7 |
| | solution | | | | | | | | | |

As shown in Table 6, about 54% of the PEG3500 was recovered from the supernatant using the TFF membrane system.

TABLE 6
PEG recovered in the different TFF fractions.

| | | Abs. | PEG | Total PEG | Volume | Total PEG | Recover from | Recover % |
|---|---|---|---|---|---|---|---|---|
| Sample | Dilution | 510 nm | (mg/L) | (mg/L) | (L) | (mg) | feed % | from T0 |
| TFF Feed | 50 | 0.511 | 32 | 1604 | 1.5 | 2406 | x | 43.0 |
| TFF Retentate | 50 | 0.770 | 48 | 2408 | 0.54 | 1301 | 54 | 23.2 |
| TFF Permeate | 50 | 0.144 | 9 | 464 | 1.05 | 488 | 20 | x |
| TFF wash | 50 | 0.040 | 3 | 141 | 3.6 | 509 | 21 | x |

Not only was PEG recovered from the supernatant, but a large majority of the beta-Glucosidase enzyme was also recovered, as shown in Table 7.

TABLE 7
Beta-Glucosidase recovered in the different TFF fractions.

| | pNPG U/ml | Volume (L) | Total BG (U) | BG recovery % |
|---|---|---|---|---|
| Feed | 3.37 | 1.5 | 5055 | |
| Retentate | 7.94 | 0.54 | 4288 | 84.8 |
| Permeate | 0.16 | 1.05 | 168 | 3.3 |
| Wash | 0.24 | 3.6 | 864 | 17.1 |

Similar results were obtained using pretreated bagasse that was subject to saccharification treatment under the same conditions as above, except that PEG8000 was added to the hydrolysis mixture instead of PEG3500. In this experiment, about 65% of the PEG8000 was recovered from the supernatant (data not shown).

In summary, the above example demonstrates that PEG can be recovered using a TFF membrane system.

### Example 5

This example demonstrates that recycled retentate comprising PEO can increase the saccharification efficiency of bagasse.

Methods: Bagasse (final solids 8.5%) was pretreated at 175° C. for 60 min. After pretreatment, the pH of the material was adjusted to pH 5.5. Retentate (2%) from TFF processed bagasse supernatant (treated with 2% PEO and 20% Accellerase® Trio™) comprising about 2% PEO (based on dry material) was added, and saccharification was performed using 20%, 15% or 10% of Accellerase® Trio™ loading (based on glucan content) for 48 hours at 50° C. Control samples did not contain retentate, or contained 3% PEG. Samples were analyzed by HPLC at time zero (T0), after 24 hours (T24) and after 48 hours (T48).

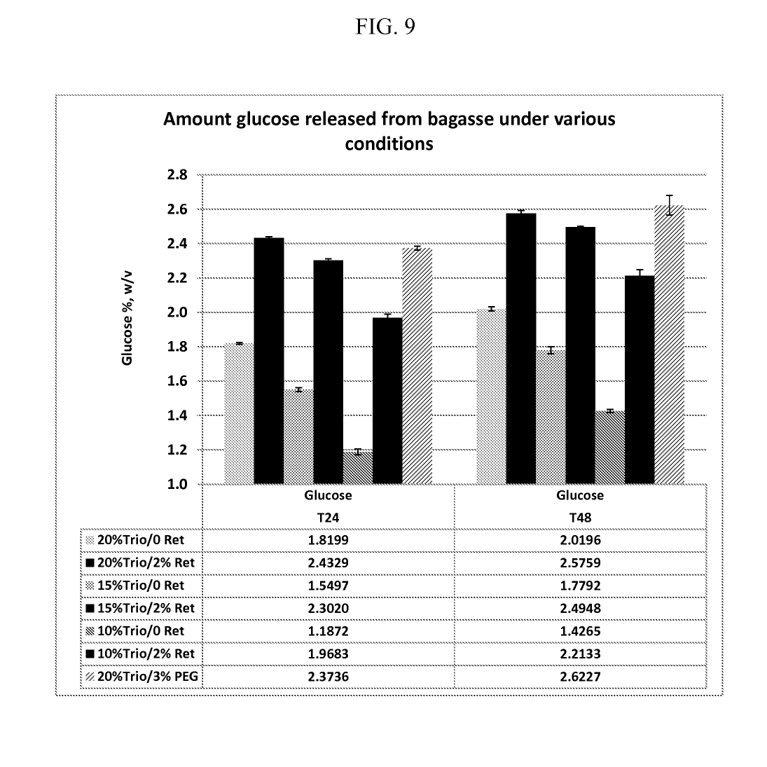

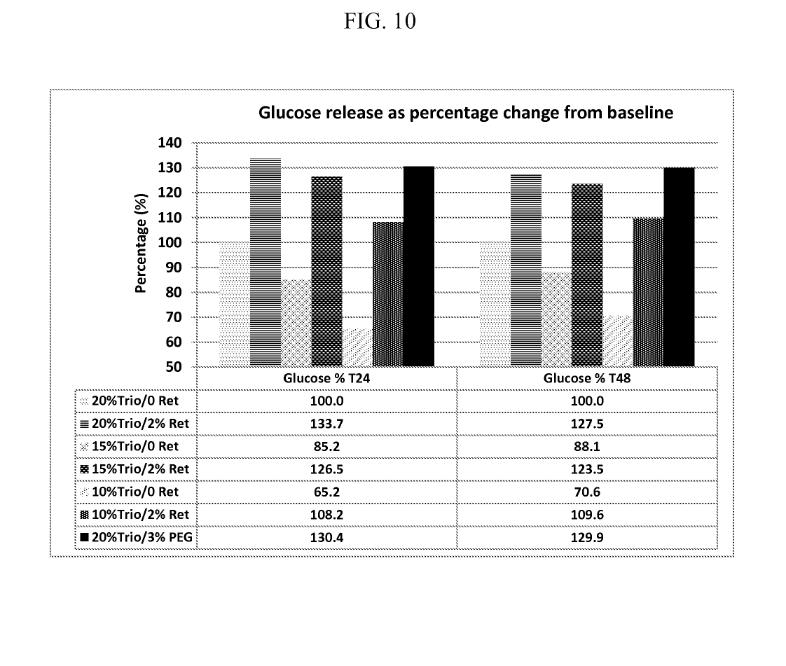

As shown in FIG. 9, the addition of 2% retentate increased glucose yield in all samples at both T24 and T48. In particular, 2% retentate plus 10% Accellerase® Trio™ resulted in about a 10% increase in glucose yield compared to 20% Accellerase® Trio™ with no retentate added (FIG. 10). Thus, the addition of 2% retentate comprising PEO can reduce enzyme usage by 50% (from 20% loading to 10% loading). 2% retentate plus 20% Accellerase® Trio™ produced a similar glucose yield as 20% Accellerase® Trio™ plus 3% fresh PEG (FIGS. 9 and 10).

This example demonstrates that recycling the retentate from a saccharification reaction comprising PEO can substantially reduce the amount of enzyme required to yield the same amount of glucose.

### Example 6

This example shows that pretreatment of corn stover with PEG increased the yield of fermentable sugars.

Methods: Corn stover was pretreated in a 1 L bioreactor comprising 18% slurry at 175° C., 30 minutes, with and without 3.0% PEG 3350 (based on dry material). Samples were treated with 20% Accellerase® Trio™ (loading based on glucan), 10% C-TecII (loading based on glucan) and 0.5% H-TecII (based on dry material). Saccharification was controlled at 50° C. for 48 hrs. Samples were taken at T0, T24 and T48 hours and analyzed by HPLC.

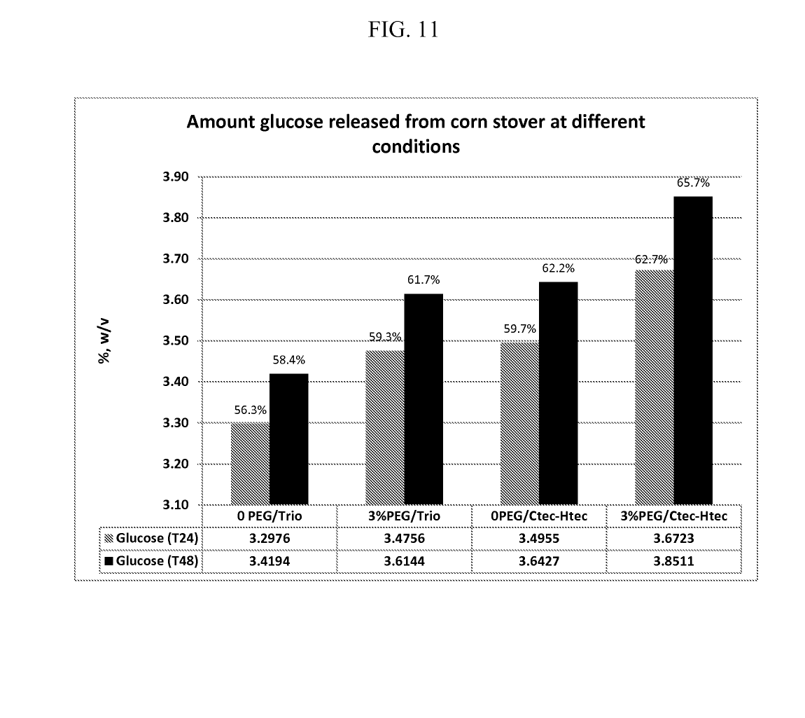

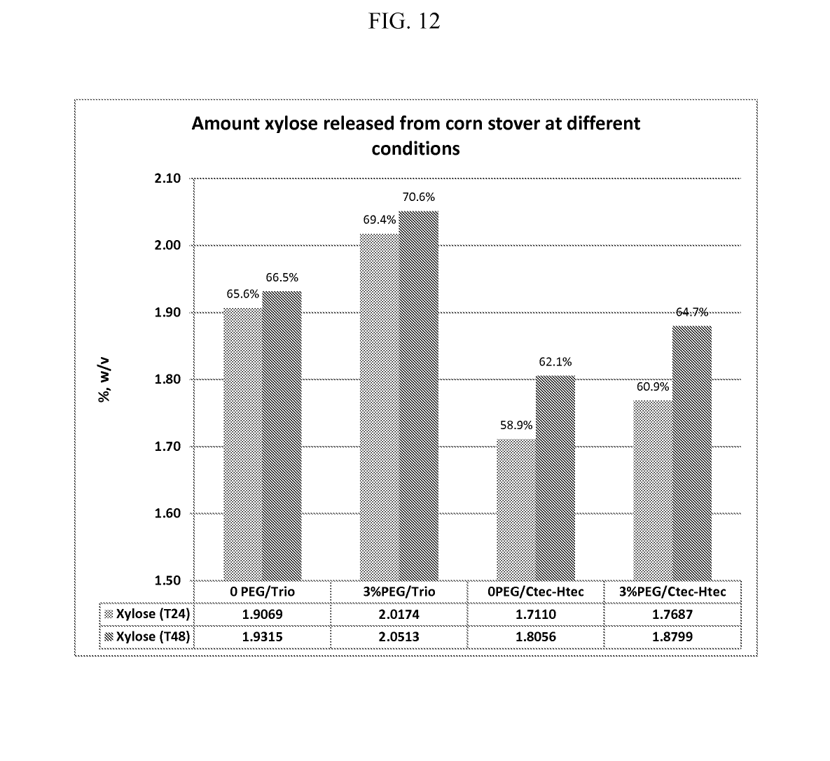

As shown in FIGS. 11 and 12, pretreatment of corn stover with PEG 3350 increased the yield of both glucose and xylose at 24 and 48 hours.

### Example 7

This example shows that treatment of bagasse with the non-ionic organic polymer PVP during saccharification increases the yield of sugars.

Standard HPHT pretreated bagasse (10% solid, pH 5.1) was treated with varying concentrations (0, 0.5, 1.0, 2.0 and 3.0% based on dry material) of PVP for 5 min, then saccharification enzymes were added (20% of Accellerase® Trio™ based on glucan content), and incubated for 48 hours at 50° C.

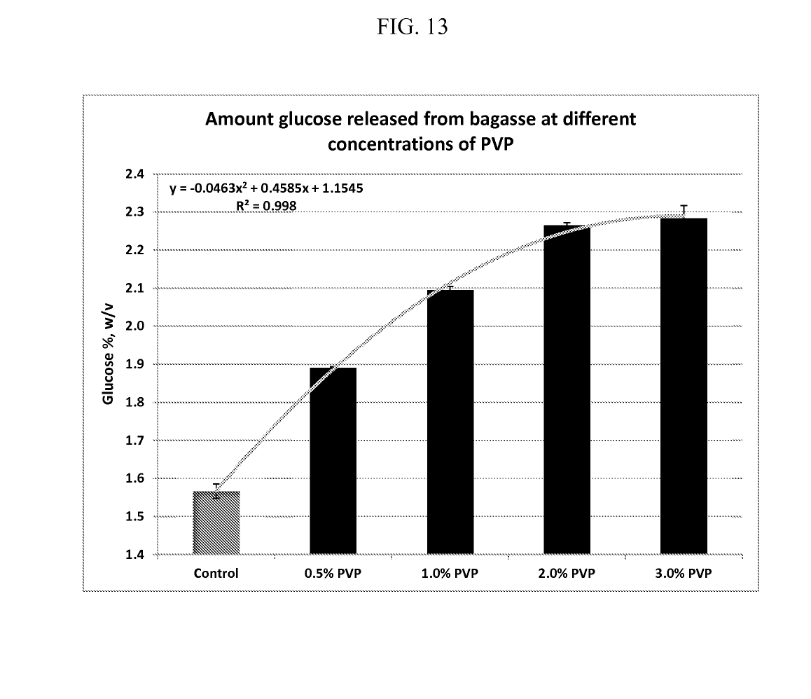

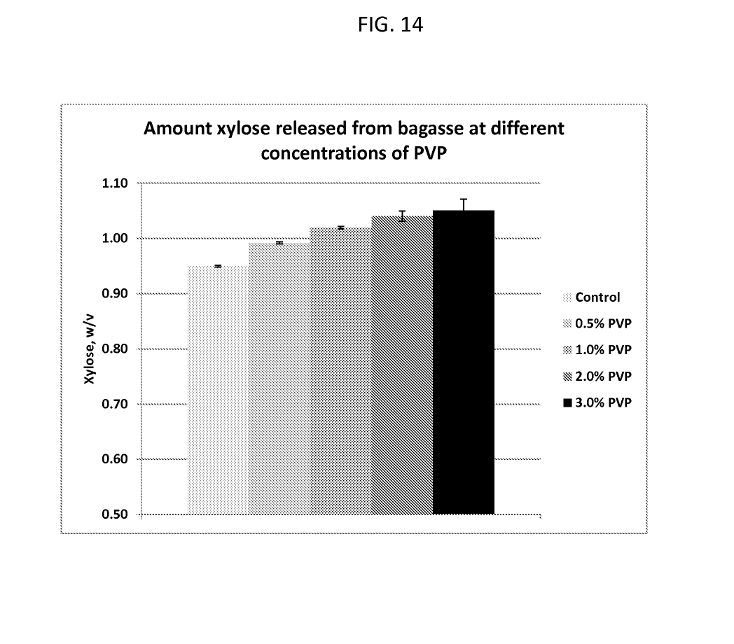

As shown in FIG. 13, treatment with 2% of PVP increased the glucose yield from the pretreated bagasse solution by over 44.6% (Table 8). PVP treatment also increased the yield of xylose (FIG. 14). Treatment with PVP did not significantly change the amount of inhibitors produced during saccharification (data not shown).

TABLE 8
Percentage increase in glucose and xylose yield from bagasse after treatment
with various concentrations of PVP (Glucan: 27%, Xylan: 15%.).

| | T24 | | | | T48 | | | |
|---|---|---|---|---|---|---|---|---|
| | Glucose | Xylose | Glucose % | Xylose % | Glucose | Xylose | Glucose % | Xylose % |
| Control | 1.5668 | 0.9499 | 100.0 | 100.0 | 1.721433 | 0.9752 | 100.0 | 100.0 |
| 0.5% PVP | 1.8926 | 0.9917 | 120.8 | 104.4 | 2.0688 | 1.0230 | 120.1 | 104.9 |
| 1.0% PVP | 2.0954 | 1.0193 | 133.8 | 107.3 | 2.2844 | 1.0510 | 132.7 | 107.8 |
| 2.0% PVP | 2.2883 | 1.0401 | 144.5 | 10 [symbol] .5 | 2.4554 | 1.0702 | 142.6 | 109.7 |
| 3.0% PVP | 2.2839 | 1.0908 | 145.8 | 110.6 | 2.4889 | 1.0700 | 144.6 | 109.7 |
| 1% PVP + 1% PEG | 2.1939 | 1.0297 | 140.1 | 108.4 | 2.3837 | 1.0581 | 138.5 | 108.5 |
| 20% PEG | 2.18 [symbol] 1 | 1.0290 | 139.5 | 108.3 | 2.3608 | 1.0628 | 137.1 | 109.0 |

[symbol]

indicates data missing or illegible when filed

### Example 8

This examples shows the effect of bagasse treated with different molecular weights of PVP on glucose and xylose yield.

Pretreated bagasse, 10% solid, pH 5.1, was incubated with 2% of different molecular weights of PVP: PVP 10K, PVP 40K and PVP 336K. 20% of Accellerase® Trio™ (based on glucan) was loaded. Saccharification was performed at 50° C. for 48 hours. Samples were taken at T0, T24 and T48 hours and analyzed by HPLC.

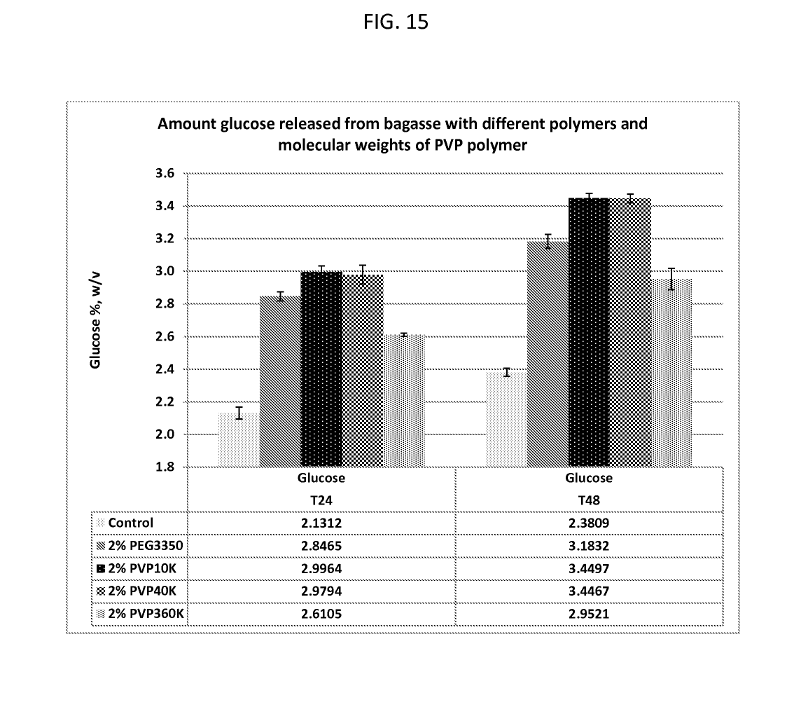

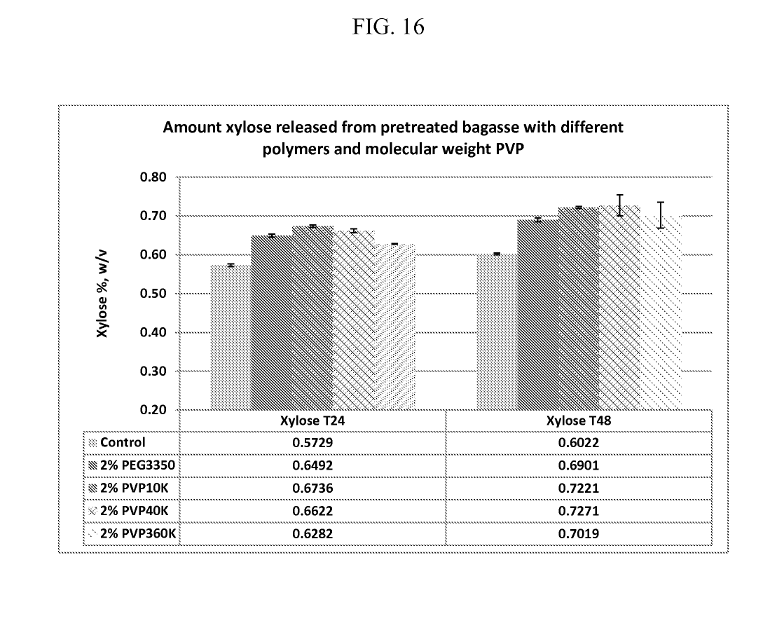

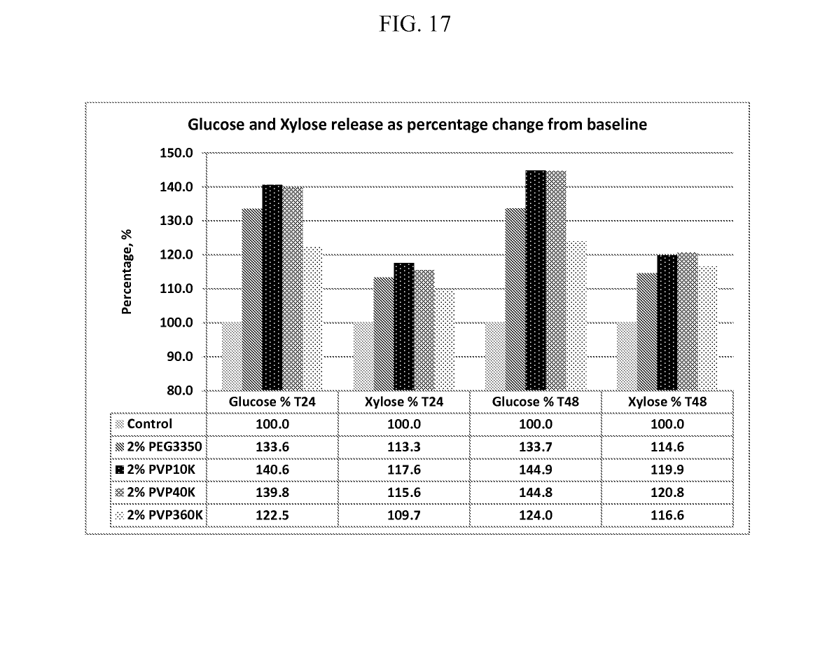

As shown in FIGS. 15 and 16, treatment of bagasse with different MW of PVP resulted in an increase in glucose and xylose yields. FIG. 17 shows that the glucose yield was increased about 40% and 45% by both 10K and 30K PVP compared to controls at T24 and T48, respectively. Further, PVP10K and PVP40K showed better results than PVP336K. The reason for the lower yield using PVP336K could be the decreased solubility and increased viscosity of higher MW PVP.

### Example 9

This example demonstrates that treatment of bagasse with both PVP and PEG increases sugar yield more than either polymer alone.

Bagasse (10% solids) was pretreated at 180° C., for 30 min, with (Bagasse #2) and without (Bagasse #1) 2% PVP (based on dry material content). Saccharification was at pH 5.0, 50° C., for 48 hours with or without the addition of 2% PVP and 2% PEG 3350 (Table 9). At T0, T24, T48 hours, samples were removed to measure concentrations of sugars and inhibitors by HPLC.

TABLE 9
Experimental design to test the effects of PVP
plus PEG on saccharification efficiency.

| Flask# | Bagasse #1 | Bagasse #2 | PVP | PEG3350 |
|---|---|---|---|---|
| 1 | 100 | | 0 | |
| 2 | 100 | | 0 | |
| 3 | 100 | | 0 | |
| 4 | 100 | | 2%, 2 ml | |
| 5 | 100 | | 2%, 2 ml | |
| 6 | 100 | | 2%, 2 ml | |
| 7 | | 100 | | |
| 8 | | 100 | | |
| 9 | | 100 | | |
| 10 | | 100 | | 2%, 0.2 g |
| 11 | | 100 | | 2%, 0.2 g |
| 12 | | 100 | | 2%, 0.2 g |

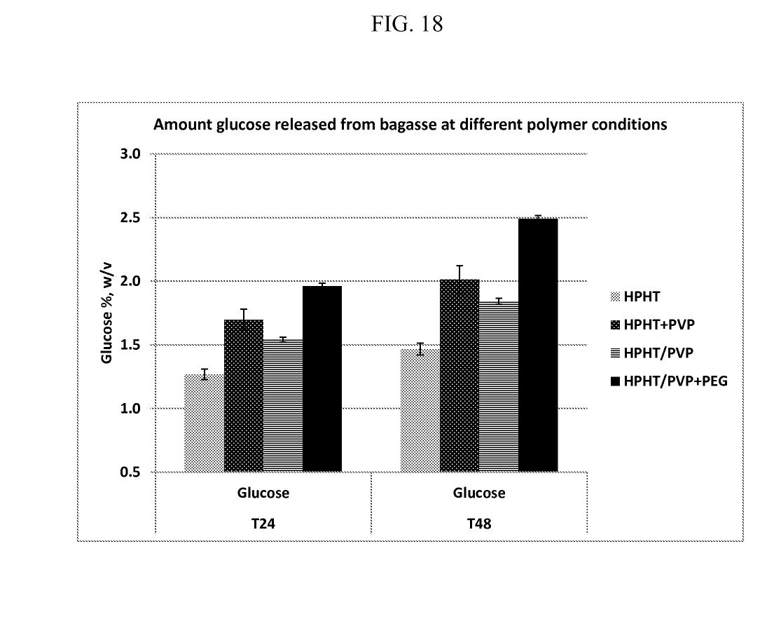

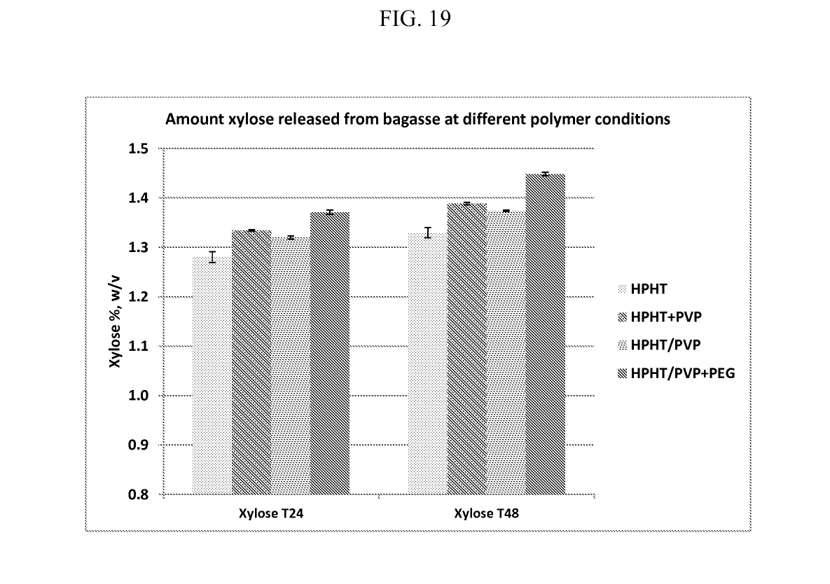

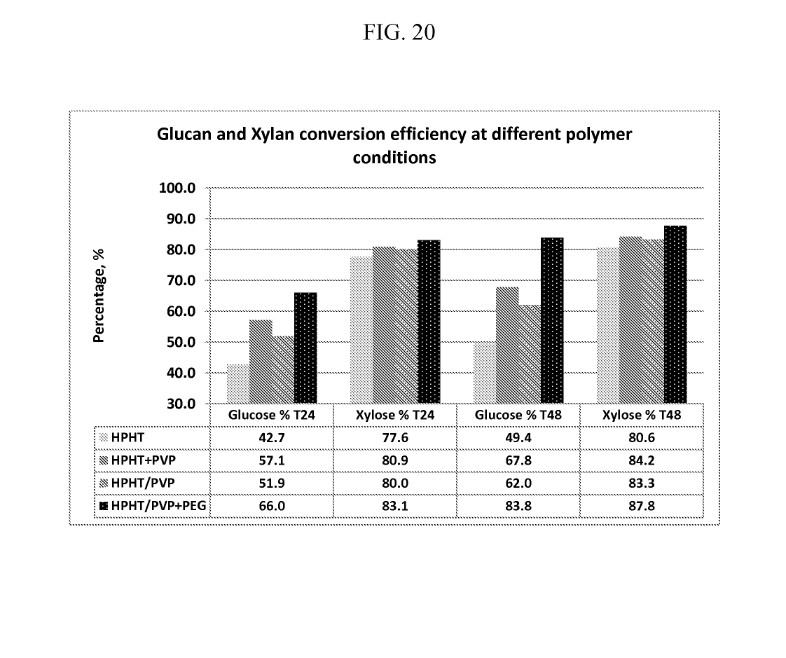

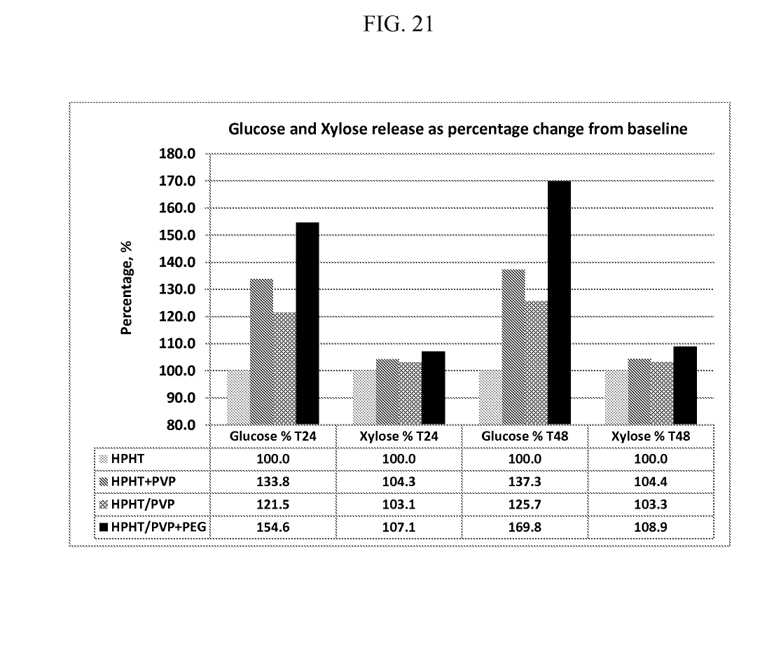

As shown in FIGS. 18 and 19, the combination of PVP plus PEG increased the yield of glucose and xylose at both 24 and 48 hours. HPHT+PVP indicates that PVP was added during saccharification. HPHT/PVP indicates PVP was added during the pretreatment step. FIG. 20 shows that pretreatment with PVP plus treatment with PEG during saccharification increased the glucose and xylose conversion rates at T24 and T48. FIG. 21 shows that pretreatment with PVP plus treatment with PEG during saccharification resulted in a 54.6% increase in glucose yield at T24, and a 69.8% increase in glucose yield at T48.

In summary, this example demonstrates that PVP added during saccharification produced higher glucose yields than PVP added during pretreatment (e.g., 33.8% vs 21.5% at T24, see FIG. 21). Moreover, the combination of pretreatment with PVP and PEG treatment during saccharification resulted in an increase in glucose yields compared to PVP treatment alone.

### Example 10

This example demonstrates that PVP treatment can reduce the amount of saccharification enzymes required to produce similar sugar yields.

Pretreated bagasse (10% solid, pH 5.1) was incubated with 20% of Accellerase® Trio™ only (control); 2% of PVP 10K with a concentration series of Accellerase® Trio™ (5%, 10%, 15% and 20%—loading based on glucan). Saccharification was performed at 50° C. for 48 hours. Samples were taken at T0, T24 and T48 hours and analyzed by HPLC.

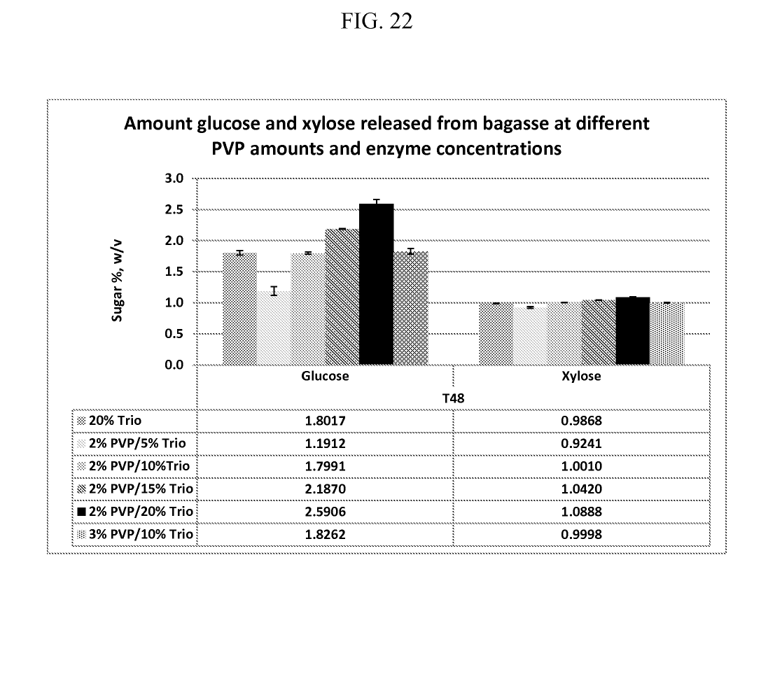

As shown in FIG. 22, at fixed PVP concentrations, more enzyme loading produced more glucose release. Treatment with 2% PVP and 10% enzyme produced similar glucose yields as 20% of enzyme without PVP treatment (FIG. 22 and Table 10). As shown in Table X, treatment with 2% of PVP plus 15% enzyme resulted in 21.4% more glucose than 20% of enzyme alone, and treatment with 2% of PVP plus 20% enzyme resulted in 43.8% more glucose than 20% of enzyme alone.

TABLE 10
Sugar yield percentage increase under
different treatment conditions

| | T48 | | | |
|---|---|---|---|---|
| | Glucose | Xylose | Glucose % | Xylose % |
| 20% Trio | 1.8017 | 0.9868 | 100.0 | 100.0 |
| 2% PVP/5% Trio | 1.1912 | 0.9241 | 66.1 | 93.6 |
| 2% PVP/10% Trio | 1.7991 | 1.0010 | 99.9 | 101.4 |
| 2% PVP/15% Trio | 2.1870 | 1.0420 | 121.4 | 105.6 |
| 2% PVP/20% Trio | 2.5906 | 1.0888 | 143.8 | 110.3 |
| 2% PVP/10% Trio | 1.8262 | 0.9998 | 101.4 | 101.3 |

In summary, this example demonstrates that treatment with PVP can reduce enzyme usage by about 50%.

### Example 11

This example shows that treatment of bagasse with polymers containing a polyvinyl structure increased saccharification efficiency.

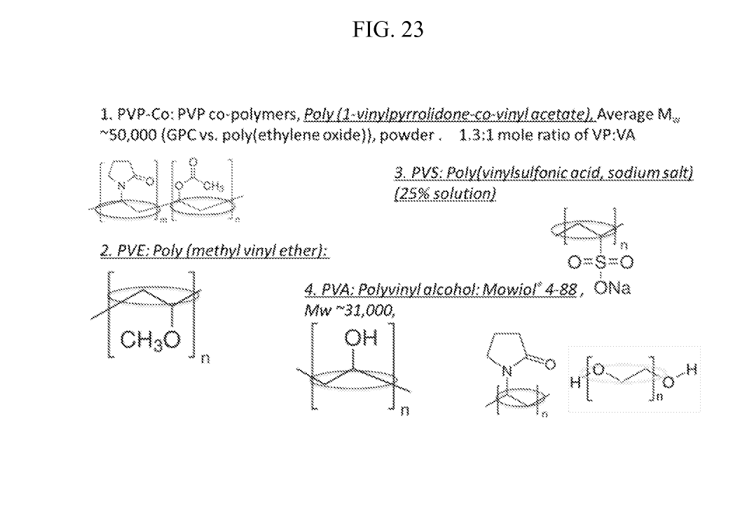

Pretreated bagasse (10% solid, pH 5.1) was incubated with 20% of enzyme loading (based on glucan), and 2% of various polymers (PVP, PVP-co polymer, PVE, PVA and PVS; the polymer structure details are shown in FIG. 23). Saccharification was performed at 50° C. for 48 hours. Samples were taken at T0, T24 and T48 hours and analyzed by HPLC.

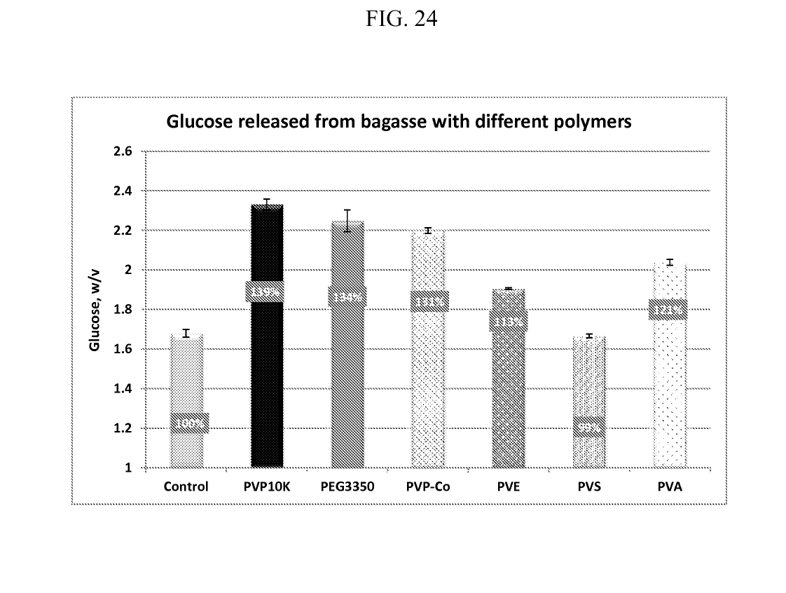

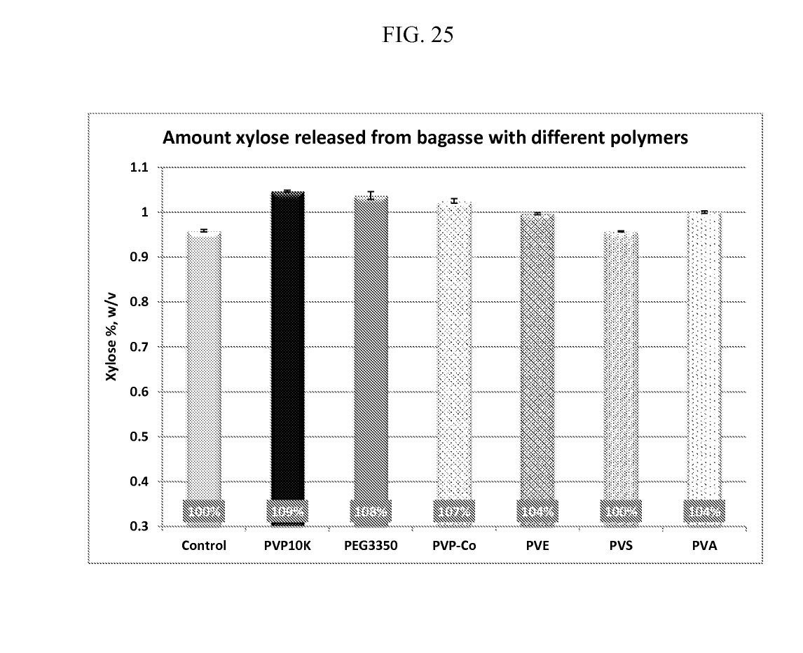

As shown in FIG. 24, after 24 hours of saccharification, the samples treated with PVP, PVP-co polymer, PVA, and PVE all increased glucose yield. All of the polymers tested had less effect on xylose yield (FIG. 25), and no effect on inhibitor release (Table 11).

TABLE 11
Inhibitors released from pretreated bagasse solution treated with different polymers.

| | T24 | | | | | | | | |
|---|---|---|---|---|---|---|---|---|---|
| | [symbol] urolic | | | | [symbol] yringic | | | [symbol] oumaric | [symbol] erulic |
| PVP-Co | acid | 5-HMF | Furfural | 4HBA | aci [symbol] | Vanillin | [symbol] yringaldehy [symbol] | ac [symbol] | aci [symbol] |
| Control | 38.7 | 70.6 | 1427.2 | 43.7 | 18.7 | 39.5 | 49.5 | 16.4 | 33.1 |
| PVP10K | 38.3 | 70.2 | 1417.0 | 43.6 | 18.6 | 39.6 | 49.1 | 17.6 | 24.0 |
| PEG3350 | 38.5 | 64.7 | 1432.4 | 45.6 | 18.9 | 38.0 | 53.2 | 18.4 | 23.6 |
| PVP-Co | 38.4 | 70.3 | 1435.6 | 42.5 | 18.3 | 35.8 | 48.8 | 17.3 | 24.7 |
| PVE | 38.6 | 64.8 | 1439.0 | 43.6 | 18.5 | 36.2 | 51.5 | 17.7 | 21.3 |
| PVS | 38.7 | 69.5 | 1400.0 | 44.9 | 19.1 | 37.6 | 53.0 | 17.9 | 20.9 |
| PVA | 38.8 | 64.7 | 1430.4 | 45.6 | 19.0 | 37.9 | 54.6 | 18.2 | 22.6 |

[symbol]

indicates data missing or illegible when filed

This example demonstrates that saccharification treatment with polymers comprising a polyvinyl structure increased the yield of glucose from pretreated bagasse.

### Example 12

This example shows that treatment with PVP increased the yield of sugars from different cellulosic biomass feedstocks.

Standard HPHT pretreated switch grass and almond shell (pretreated at 180° C., 20% solid, pH 5.1) and dilute acid pretreated corn stover (0.1% of H2SO4, HPHT at 180° C., 30 min, 15% solid, pH 5.0) were used for these experiments. Saccharification enzymes were added (20% of Accellerase® Trio™ based on glucan content), and incubated for 24 to 48 hours at 50° C.

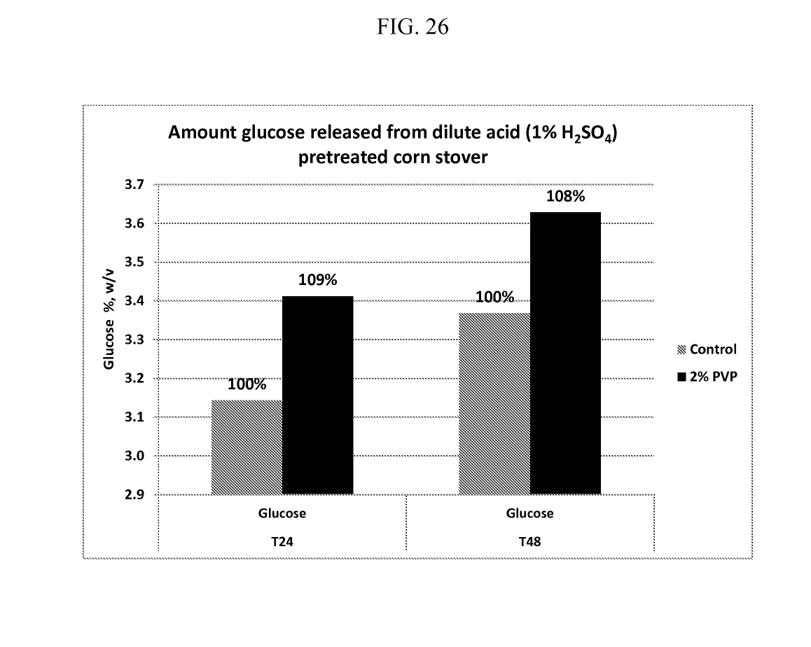

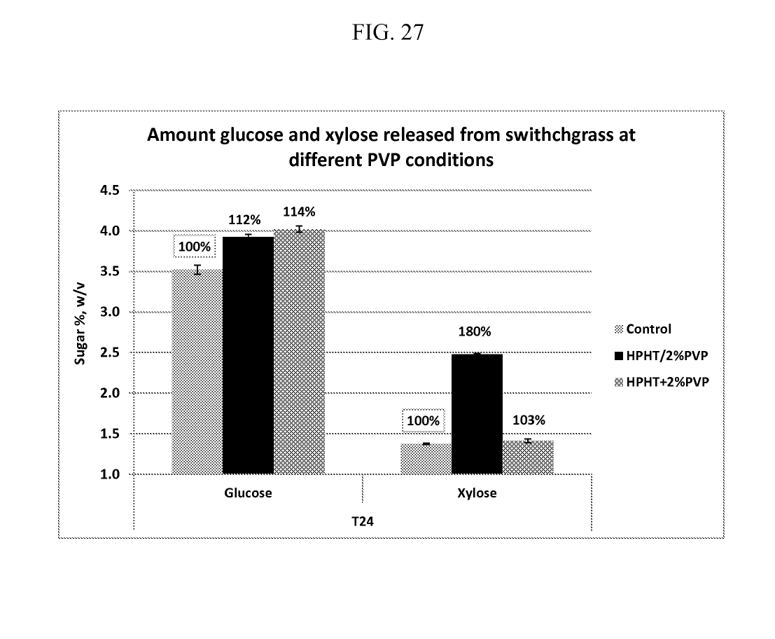

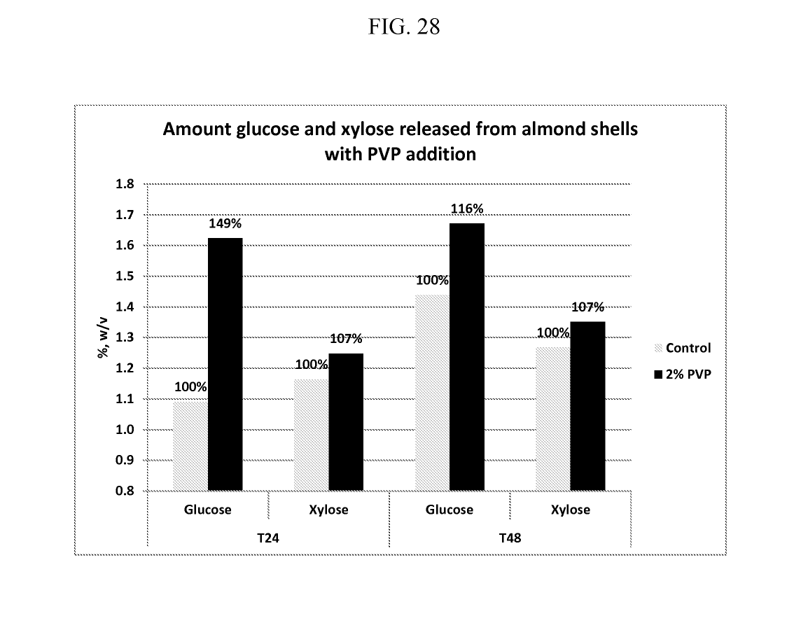

As shown in FIGS. 26 and 27, treatment with 2% PVP increased the glucose yield from pretreated corn stover and switch grass by about 8-14%. As shown in FIG. 28, treatment of pretreated almond shell solution with 2% PVP increased the glucose yield by 49% at T24 and 16% at T48 compared to controls (no PVP treatment). Xylose yields were also increased, but not to the same extent as glucose.

This example demonstrates that treatment with PVP can increase sugar yields from a variety of different cellulosic biomass feedstocks.

### Example 13

This example describes a system that integrates continuous biomass saccharification with recycling of a non-ionic organic polymer and recycling of enzymes.

FIG. 29 shows a flow chart for a continuous saccharification system that is combined with a TFF membrane system and recycling of the retentate. Cellulosic biomass, such as bagasse, is mixed with an aqueous fluid and subjected to HPHT pretreatment. The HPHT pretreatment can occur in a high shear mixing device such as an auger. After pretreatment, the pretreated biomass is hydrolyzed in a saccharification reactor. The saccharification reactor can be a high shear mixing device, such as an auger. Saccharification enzymes and a non-ionic organic polymer, such as PEO or PVP, is added to the saccharification mixture. If desired, a series of saccharification reactors can be used (labeled (A) to (X), where X is an integer). Following saccharification, the liquefied biomass is separated into a liquid stream and a solids stream using a solid/liquid separation system or device, as described herein. The solids stream can be recycled back to a saccharification reactor. The solids can further comprise enzymes that are recycled back to a saccharification reactor, where the recycled enzymes increase the efficiency of saccharification and reduce the amount of fresh enzymes that are required for saccharification, thereby reducing the expense of fresh enzymes. The liquid stream is further separated into a retentate and permeate using a filter system, such as a TFF system. In the embodiment shown in FIG. 29, the permeate comprising sugars is sent to a fermentation tank for the production of ethanol. Alternatively, the permeate and sugars can be used for any desired downstream purpose. The retentate comprising the non-ionic organic polymer and enzymes is recycled back to a saccharification reactor. The recycled retentate further improves the saccharification efficiency and reduces the amount of fresh enzymes required, providing a cost savings to the ethanol plant operator.

### Example 14

This example demonstrates that polymers could be concentrated and separated from a process stream comprising sugars and thereby recycled.

Material and Methods

A. Polymer and Water Testing

The polymers tested were polyvinyl alcohol (PVA) and polyvinylpyrrolidone (PVP). Aqueous polymer solutions (2% w/w) were made by mixing the polymer with water and heating to 70° C. The polymers were concentrated using an OptiSep 1000 TFF filter (SmartFlow Technologies, Apex, N.C.) containing a membrane with a 20 kDa polyether sulfone (PES) membrane. The solutions were concentrated to a 4× concentration. Samples were taken of the feed material and of the retentate and permeate pool at 2× and 4× concentrations. These samples were assayed for polymer concentrations using the assay described below.

B. Polymer Concentration Assay

The PVP concentration was determined using a colorimetric UV-Vis absorbance method. This assay employs Congo Red dye and an absorbance shift measured when PVP is added to the dye. 25 μL of each sample was added to 5 ml of Congo red working solution made by dissolving 0.1 g Congo Red in 100 ml of water. The absorbance of the mixture was measured at 500 nm and compared to a standard curve.

The PVA concentration was determined using a colorimetric UV-Vis absorbance method. This assay utilizes the formation of a blue complex that PVA forms with Boric acid and tri-iodide. 1 mL of each sample was added to 24 ml of reverse osmosis (RO) water. 15 ml of 0.65 M boric acid solution was added to the sample. Finally, 3 ml of KI/Iodine solution (0.1506 M KI and 0.05 M Iodine) and 7 ml of water are then added to the mixture. The absorbance of the mixture was measured at 690 nm and compared to a standard curve.

C. Polymer and Saccharification Material Testing

Bagasse material was pretreated by heating to 178° C. for 30 minutes. The designated polymer (either PVA or PVP) was dosed in the slurry at a concentration of 2% (w/w) with respect to the biomass solids in the solution. An enzyme cocktail (Accellerase Trio from DuPont), was added to the biomass slurry at a concentration of the 20% w/w with respect to the β-glucan in the biomass. The slurry was permitted to undergo hydrolysis for 72 hours. At this point, the resulting solution was passed through a 25 um vibrating sieve (SWECCO, Florence, Ky.). The effluent was transferred to the TFF system as described above. The material was concentrated to a final concentration of 3×. Samples were taken of the feed material and of the retentate and permeate pool at 2× and 4× concentrations. These samples were assayed for polymer concentrations using the assay described above.

D. Enzyme Recycle with and without Polymers

The effect of polyethylene glycol (PEG) on β-glucosidase enzyme recycle was tested by measuring the enzyme activity with and without PEG on two batches of biomass. The first batch contained newly pretreated biomass while the second batch contained newly pretreated biomass and recycled solids, enzymes, and PEG from a solid/liquid separation of a partially hydrolyzed batch of biomass as described below. For the first batch, roughly 100 kg of biomass and water solution at 10% solids was pretreated by heating to 178° C. for 30 minutes. In one experiment PEG was dosed in the slurry at a concentration of 2% (w/w) with respect to the biomass solids in the solution while in the control experiment PEG was not used. An enzyme cocktail was added to the biomass slurry at a concentration of the 20% w/w with respect to the β-glucan in the biomass. The slurry was permitted to undergo hydrolysis for 16 hours. At this point, the resulting solution was passed through a 25 um vibrating sieve (SWECCO, Florence, Ky.). The effluent was transferred to the TFF system containing OptiSep 7000 membrane modules. A total of 1.8 m² of PES membrane with a 150 kDa pore size was used to process the batch. The material was concentrated to a 3× concentrate. A sample of the concentrate was taken, and it was assayed in the method described below to determine the remaining β-glucosidase (BG) enzyme activity in relation in the original enzyme dosed into the material. To create the second batch, the material that did not pass through the vibrating sieve and the TFF retentate were recombined with fresh biomass that was pretreated in the same method as was described above. Fresh enzymes were added to recombined slurry at a dosing of 20% w/w with respect to the “fresh” glucan that was added to the tank. The slurry was hydrolyzed for 16 hours. Then the material was processed through the vibrating sieve and TFF system using the same method as the first batch.

β-glucosidase activity was measured using a pNPG microplate assay. The pNPG assay is an initial rate assay in which p-nitrophenyl-β-D-glucoside (pNPG) substrate is converted to p-nitrophenol (pNP) by β-glucosidase enzyme. The biomass hydrolysate samples were centrifuged at 4600×G for 10 minutes to separate the solid and liquid phases. After centrifugation, the supernatant was removed from the solid pellet via pipette. The solid pellet was suspended in 125 mL of 50 mM sodium acetate buffer containing 0.5% Tween 80 (pH 5.3) and incubated for two hours at 44° C. and 200 rpm to desorb any enzyme bound to the surface of the biomass substrate. Suspended solids were allowed to settle after incubation, after which 40 mL of supernatant was collected. The liquor samples and buffer samples were centrifuged again (4600×G for 10 minutes) to remove any solids before a two-stage diafiltration. Samples (4 mL each) were centrifuged at 4600×G for 30 minutes using MicroSep Advanced Centrifuge tubes (10 kDa). After the first round of centrifugation, retentate volume was made-up to 4 mL using 200 mM sodium acetate buffer (pH 5.0) and the samples were centrifuged at 4600×G for an additional 30 minutes. Filter permeate was discarded between centrifugation steps. The exact volume of retentate was recorded after diafiltration.

The pNPG assay was performed in a 96-well microplate. Enzyme samples from the liquid and solid phases were diluted as necessary before the assay. Dilute enzyme aliquots were combined with equivalent amounts of 200 mM sodium acetate buffer and RO water (25 μL each). The enzyme solution and a 10 mM pNPG solution were incubated for 5 minutes at 50° C. before 25 μL of pNPG solution was added to the enzyme solution to initiate the reaction. Samples were incubated for 10 minutes at 50° C., and then the reaction was terminated by adding 100 μL of 250 mM sodium carbonate. Reacting samples, as well as blanks for enzyme, buffer, and substrate, were tested in duplicate. Sample absorbance was measured at 405 nm to determine the amount of pNP produced based on a biomass-specific calibration curve. Each μmol pNP produced per minute corresponds to one unit of β-glucosidase activity. Total β-glucosidase activity per unit volume of a biomass hydrolysate sample was calculated based on the enzyme retentate volumes of the solid and liquid phases, and the mass ratio of solid pellet to liquid supernatant after initial centrifugation.

Results

E. Polymer and Water Testing

Table 12 displays the concentration of the PVP in the feed material (1× concentration) and the concentrate and permeate pool at 2× and 4× concentration factors. The PVP was retained by the membrane as its measured concentration increased by 3.69×, which is 92% of the maximum theoretical 4× concentration. The remaining material was measured in the permeate, which means that a small fraction of the polymer passed through the membrane. Additionally, when a mass balance was performed on the PVP, 92% of the material was recovered in the retentate. Therefore, the PVP can be concentrated and recycled using a TFF membrane.

TABLE 12
PVP concentration in concentrate and permeate pool during
a 4X concentration using PVP mixed with water.

| | Concentrate | Permeate Pool |
|---|---|---|
| Conc | PVP %(w/v) | PVP %(w/v) |
| 1 | 2.16 | |
| 2 | 3.82 | 0.88 |
| 4 | 7.97 | 0.96 |

Table 13 displays the concentration of the PVA in the feed material (1× concentration) and the concentrate and permeate pool at 2× and 4× concentration factors. The PVA was well retained by the membrane as its measured concentration increased by 3.27×, which is 82% of the maximum 4× concentration. The remaining material was measured in the permeate, which means that a small fraction of the polymer passed through the membrane. Additionally, when a mass balance was performed on the PVA, 81% of the material was recovered in the retentate. Therefore, the PVA can be concentrated and recycled using a TFF membrane.

TABLE 13
PVA concentration in concentrate and permeate pool during
a 4X concentration using PVA mixed with water.

| | Concentrate | Permeate Pool |
|---|---|---|
| Conc | PVA (%) w/v | PVA (%) w/v |
| 1 | 1.93 | |
| 2 | 3.11 | 0.0517 |
| 4 | 6.32 | 0.0393 |

F. Polymer and Saccharification Material Testing

After demonstrating that the PVP and PVA can be concentrated using the TFF filter, the process was modified to determine if the PVP and PVA could be recycled with biomass. Table 14 shows the concentration of PVP with biomass present. In the case of PVP, the polymer did not concentrate as effectively with the biomass present as without the biomass. However, the PVP did concentrate to 1.6× its initial concentration which led to a recovery of 54% of the polymer that was fed to the TFF system. This low recovery was due to 29% of the PVP passing through the filter and additional 18% of the polymer that was lost in the system. These losses may be due to binding of the material to the filter. Additionally, it should be noted that only 13% of the PVP that was dosed to the system was present in the TFF feed. These losses may be due to binding with material (such as lignin, cellulose, and hemicellulose) in the reaction mixture. Overall, these results demonstrate that PVP can be recycled with biomass present.

TABLE 14
PVP concentration in concentrate and permeate pool during
a 3X concentration using PVP mixed with biomass.

| | Concentrate | Permeate Pool |
|---|---|---|
| Conc | PVP %(w/v) | PVP %(w/v) |
| 1 | 0.03 | |
| 2 | 0.04 | 0.010 |
| 3 | 0.04 | 0.011 |

Table 15 shows the concentration of PVA with biomass present, and demonstrates that PVA was readily concentrated with the biomass present. The PVA concentrated to 3.17× its initial concentration which led to a recovery of 105% of the polymer that was fed to the TFF system. In the case of the PVA, 86% of the PVA that was fed into the system was present in the TFF feed. These results demonstrate that PVA can be recycled with biomass present.

TABLE 15
PVA concentration in concentrate and permeate pool during
a 3X concentration using PVA mixed with biomass.

| | Concentrate | Permeate Pool |
|---|---|---|
| Conc | PVA %(w/v) | PVA %(w/v) |
| 1 | 0.17 | |
| 2 | 0.35 | 0.003 |
| 3 | 0.55 | 0.003 |

G. Enzyme Recycle with and without Polymers

Table 16 displays the fraction of the original BG that was present in the 3×TFF concentrate. The enzyme concentration was only a fraction (roughly 0.4 times) of the initial enzyme concentration without the polymer present. However, when the polymer was added to the solution, the fraction jumped to 1.6 times the initial dosing. Additionally, this fraction was fairly consistent over both batch 1, which contained only newly pretreated biomass, and batch 2, which contained newly pretreated biomass and recycled solids, enzymes, and PEG from a solid/liquid separation from a partially hydrolyzed batch of biomass. Therefore, adding polymer greatly increased the fraction of enzyme that is available for recycle. Additionally, the recycle of both the polymer and the enzyme can be accomplished using the same unit operations (vibrating sieve followed by TFF).

TABLE 16
Fraction of original β-glucosidase activity in 3x
TFF concentrate both with and without PEG in the process

| | Without PEG | With PEG |
|---|---|---|
| Batch 1 | 0.47 | 1.54 |
| Batch 2 | 0.36 | 1.65 |

In conclusion, this example demonstrates that both PVP and PVA can increase the amount of enzyme that can be recovered and recycled from treated biomass.

### Example 15

This Example compares the effect of treating biomass using the Cellunator® high shear milling device after thermal pretreatment (PT) and before hydrolysis with and without PEG.

Methods

Pretreatment was performed in 40-gal batches in a pressure vessel jacketed for steam and cooling water. Bagasse was loaded at 8% solids. Aliquots of sieved bagasse were gradually fed into the tank, allowing the agitator to hydrate and suspend the dry feedstock. The tank was sealed after loading the bagasse and steam was fed into the jacket to heat the slurry to 176.7° C. (135 psi). After a 30 minute hold at the target temperature, the steam feed was shut off and drained from the jacket. Chilled process water was circulated through the jacket until the bagasse slurry was cooled to <95° C. The vessel was unsealed after the cooling phase was completed.

One batch was processed through the Cellunator after thermal pretreatment while a control batch was not processed through the Cellunator. The pretreated bagasse was treated with the Cellunator once the pretreated bagasse had cooled to 85° C. in the pretreatment vessel. Pump flow rates and times were adjusted so that the pretreated bagasse would make 5-6 passes through the MK-10 Cellunator operating at a fixed radial gap setting of 1.122 mm. A drain at the bottom of the tank fed a progressive cavity slurry pump that circulated pretreated bagasse through the Cellunator and back to the top of the pretreatment vessel.

The Cellunated and non-cellunated bagasse were saccharified at two conditions: 20% enzyme (w/w with respect to glucan) and 20% enzyme (w/w with respect to glucan)+2% PEG (w/w with respect to solids). The enzyme used was Accellerase Trio (Dupont, Palo Alto, Calif.). Deionized water was added as a blank to flasks that did not receive the full dose of enzyme or PEG so that dilution of the solids was uniform across all flasks. Before saccharification, the pH of the biomass slurry was adjusted from 3.52 to 5.47 through addition of ammonium hydroxide solution. Flasks were sampled at t=4, 24, and 48 hours. Sugar concentrations in the samples were measured via HPLC.

Results

Table 17 shows the results using Cellunator treatment in combination with PEG on the saccharification yield of bagasse. The data illustrates that Cellunator treatment increases the C₆ (glucan) yield from 52% to 56% without the use of PEG and from 77% and 81% with the use of PEG. Therefore, the combination of using both the Cellunator and PEG polymer addition resulted in the highest overall conversion.

TABLE 17
The saccharification (both C6 and C5 yields) results
of a bagasse sample both with and without treatment
with a Cellunator and with and without PEG addition.

| | | Not | Not | | |
|---|---|---|---|---|---|
| | | Cellunated, | Cellunated, | Cellunated, | Cellunated, |
| | Time | No PEG | with PEG | No PEG | with PEG |
| C6 | 0 | 1% | 1% | 1% | 1% |
| Yield | 4 | 23% | 31% | 22% | 31% |
| | 24 | 45% | 67% | 48% | 70% |
| | 48 | 52% | 77% | 56% | 81% |
| C5 | 0 | 31% | 31% | 27% | 27% |
| Yield | 4 | 56% | 59% | 52% | 55% |
| | 24 | 65% | 71% | 61% | 66% |
| | 48 | 67% | 73% | 63% | 69% |

The above example demonstrates that treating biomass with a high-shear milling device followed by hydrolysis with the addition of PEG results in the highest yields of sugars.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes. In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded.

What is claimed is:

1. A method for generating sugars from biomass, comprising:
(a) providing a mixture comprising:
the biomass;
a non-ionic organic polymer of sufficient size to be captured by a filter; and
one or more enzymes to hydrolyze components of the biomass to sugars;
(b) incubating the mixture under conditions such that the one or more enzymes hydrolyze components of the biomass to sugars, thereby producing a mixture of solids and a liquid comprising the polymer and sugars;
(c) separating the mixture into a liquid stream comprising the polymer and sugars, and a solids stream comprising solids;
(d) separating the liquid stream with the filter into a permeate comprising sugars and a retentate comprising the polymer; and
(e) returning at least a portion of the retentate to said mixture or a new mixture comprising biomass, thereby generating sugars and re-using the polymer.

2. The method of claim 1, wherein the polymer has the formula (I): 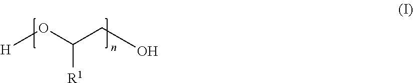
wherein R¹ is H, or a C₁₋₆ alkyl, and n is an integer greater than 1.

3. The method of claim 1, wherein the polymer has the formula (II): 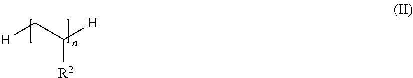
wherein R² is a hydroxyl, alkoxy, substituted or unsubstituted carboxylate, or substituted or unsubstituted heterocyclyl, and n is an integer greater than 1.

4. The method of claim 1, further comprising returning at least a portion of the solids stream to the mixture, wherein the solids stream comprises at least a portion of the one or more enzymes.

5. The method of claim 1, wherein the concentration of the polymer in the mixture is from about 0.1% to about 10.0% by weight of solids in the biomass.

6. The method of claim 2 or 3, wherein n is greater than 25.

7. The method of claim 2 or 3, wherein n is between 25 and 250,000.

8. The method of claim 1, wherein the biomass is a lignocellulosic biomass.

9. The method of claim 1, wherein the biomass comprises at least about 10% solids w/w in step (a).

10. The method of claim 1, wherein the biomass is a pretreated biomass.

11. The method of claim 1, wherein the separating (c) of the mixture comprises using a mechanical device, a filter, a membrane, or a tangential flow filtration device.

12. The method of claim 11, wherein the mechanical device is a centrifuge, a press, or a screen.

13. The method of claim 1, wherein the filter comprises a membrane or a tangential flow filtration device.

14. The method of claim 1, wherein the sugars comprise glucose and xylose.

15. The method of claim 14, wherein the yield of glucose is increased compared to a mixture that does not contain the polymer.

16. The method of claim 14, wherein the yield of xylose is increased compared to a mixture that does not contain the polymer.

17. The method of claim 1, wherein the sugars from the liquid stream in step (c) and/or the permeate from step (d) are processed into ethanol, biofuels, biochemicals, or other chemical products.

18. The method of claim 1, wherein the one or more enzymes comprise a cellulase such as exo-cellobiohydrolases, endo-gluconases, and beta-glucosidases; a hemicellulase such as xylanases, beta-xylosidases, arabinofuranosidases; starch hydrolyzing glycosidases and amylases, ligninases, and feruloyl esterases; or non-hydrolytic enzymes such as oxidoreductases and lyases.

19. The method of claim 1, wherein the mixture comprises two or more different non-ionic organic polymers.

20. The method of claim 19, wherein the two or more different non-ionic organic polymers comprise a polymer of formula (I) and a polymer of formula (II): 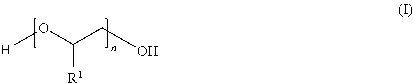
wherein R¹ is H, or a C₁₋₆ alkyl and n is an integer greater than 1; and 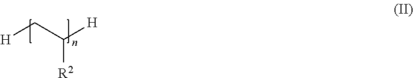
wherein R² is a hydroxyl, alkoxy, substituted or unsubstituted carboxylate, or substituted or unsubstituted heterocyclyl, and n is an integer greater than 1.

21. A method for generating sugars from biomass, comprising:
(a) contacting the biomass with a non-ionic organic polymer of sufficient size to be captured by a filter and one or more enzymes under conditions such that the one of more enzymes hydrolyze components of the biomass to sugars, thereby producing a mixture of solids and a liquid comprising the polymer and sugars, thereby generating sugars.

22. The method of claim 21, wherein the polymer has the formula (I): 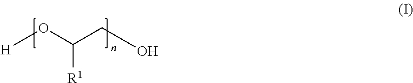
wherein R¹ is H, or a C₁₋₆ alkyl, and n is an integer greater than 1.

23. The method of claim 21, wherein the polymer has the formula (II): 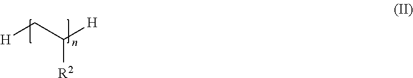
wherein R² is a hydroxyl, alkoxy, substituted or unsubstituted carboxylate, or substituted or unsubstituted heterocyclyl, and n is an integer greater than 1.

24. The method of claim 21, wherein n is greater than 25.

25. The method of claim 21, wherein n is between 25 and 250,000.

26. The method of claim 21, wherein the one or more enzymes comprises a cellulase such as exo-cellobiohydrolases, endo-gluconases, and beta-glucosidases; a hemicellulase such as xylanases, beta-xylosidases, arabinofuranosidases; starch hydrolyzing glycosidases and amylases, ligninases, and feruloyl esterases; or non-hydrolytic enzymes such as oxidoreductases and lyases.

27. The method of claim 21, wherein the mixture comprises two or more different non-ionic organic polymers.

28. The method of claim 27, wherein the two or more different non-ionic organic polymers comprise a polymer of formula (I) and a polymer of formula (II): 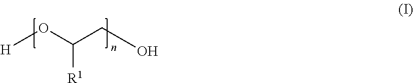
wherein R¹ is H, or a C₁₋₆ alkyl and n is an integer greater than 1; and 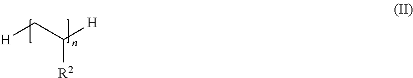
wherein R² is a hydroxyl, alkoxy, substituted or unsubstituted carboxylate, or substituted or unsubstituted heterocyclyl, and n is an integer greater than 1.

29. The method of claim 21, wherein the activity of the enzyme(s) is increased at temperatures greater than 55° C. compared to the activity of the enzyme(s) in the absence of the polymer of formula (I).

30. The method of claim 21, wherein the activity of the enzyme(s) is increased at a pH of 6.0 compared to the activity of the enzyme(s) in the absence of the polymer of formula (I).

31. The method of claim 1 or 21, further comprising:
(a) contacting the biomass with a polymer of formula (I) having an average molecular weight or an My of from about 1,000 to about 10,000,000 under conditions suitable to hydrolyze components of the biomass to sugars.
